(19) United States
(12) Patent Application Publication
(10) Pub. No.: US 2009/0217409 A1
(43) Pub. Date: Aug. 27, 2009

(54) METHODS FOR INCREASING THE FREQUENCY OF APOMIXIS EXPRESSION IN ANGIOSPERMS

(76) Inventor: John G. Carman, Smithfield, UT (US)

Correspondence Address: Utah State University;Fennemore Craig, 570 Research Park Way, Suite 101, North Logan, UT, 84341-2032, US

(21) Appl. No.: 12/416,883

(22) Filed: Apr. 1, 2009

Related U.S. Application Data
(63) Continuation of application No. 10/969,054, filed on Oct. 21, 2004.
Application No. 10/969,054 is a continuation-in-part of application No. 10/772,243, filed on Feb. 6, 2004.
Application No. 10/772,243 is a continuation-in-part of application No. 09/744,614, filed on Jan. 26, 2001, now abandoned.
Application No. 09/744,614 is a continuation-in-part of application No. 10/785,157, filed on Feb. 25, 2004, now Pat. No. 7,541,514.
Application No. 10/785,157 is a division of application No. 09/576,623, filed on May 23, 2000, now Pat. No. 6,750,376.
Application No. 09/576,623 is a continuation of application No. 09/018,875, filed on Feb. 5, 1998, now abandoned.
(60) Provisional application No. 60/512,919, filed on Oct. 22, 2003.
(60) Provisional application No. 60/162,626, filed on Oct. 29, 1999.
(60) Provisional application No. 60/037,211, filed on Feb. 5, 1997.

(51) Int. Cl. A01H 1/02

(52) U.S. Cl. 800/271

(57) ABSTRACT

The present invention is directed to the seed-to-seed perpetuation of hybrid vigor and other traits through apomixis (asexual seed formation) in flowering plants (angiosperms). More particularly, to predictable methods for producing, from sexual or facultatively-apomictic plants, progeny plants that express an increased percentage of apomictic seed set or one or more elements of apomixis. This invention uses: plant cyto-embryology procedures to identify and select a plant or group of plants that possess appropriate genetic variability for initiation times and durations of megasporogenesis (female meiosis), embryo sac formation, egg and central cell formation and maturation, fertilization, embryony and endosperm formation; plant breeding procedures to produce numerous and divergent genetically-recombined early to late generation progeny such that embryo sac formation preempts megasporogenesis and embryony preempts fertilization; and plant cyto-embryology or progeny test procedures to select segregant plants that express an increased frequency of one or more elements of apomixis.

### CROSS-REFERENCE TO RELATED APPLICATIONS

This application is a continuation of U.S. application Ser. No. 10/969,054 filed Oct. 21, 2004, claims the benefit of priority of U.S. Provisional Application No. 60/512,919, filed Oct. 22, 2003; and is also a continuation-in-part of U.S. application Ser. No. 10/772,243 filed Feb. 6, 2004, which is a continuation-in-part of U.S. application Ser. No. 09/744,614 filed Jan. 26, 2001, which is the U.S. National Stage of International Application No. PCT/US00/29905, filed Oct. 30, 2000, which claims priority to U.S. Application No. 60/162,626, filed Oct. 29, 1999; and is also a continuation-in-part of U.S. application Ser. No. 10/785,157 filed Feb. 25, 2004, which is a divisional of U.S. application Ser. No. 09/576,623 filed May 23, 2000, now U.S. Pat. No. 6,750,376, which is a continuation of U.S. application Ser. No. 09/018,875 filed Feb. 5, 1998, which claims the benefit of priority from U.S. Application No. 60/037,211 filed Feb. 5, 1997, the entire contents of each of which are expressly incorporated herein by reference thereto.

### FIELD OF THE INVENTION

This invention relates to the seed-to-seed perpetuation of hybrid vigor and other traits through apomixis (asexual seed formation) in flowering plants (angiosperms). More particularly, it provides predictable methods for producing, from sexual or facultatively-apomictic plants, progeny plants that express an increased percentage of ovules in which normal sexual development is replaced by aposporous or diplosporous (apomictic) embryo sac formation, parthenogenesis (embryo formation from an egg without fertilization), adventitious embryony (embryo formation from cells other than the egg), or endosperm formation of the autonomous (central cell not fertilized) or pseudogamous (central cell fertilized) types. It also provides descriptions of embryological phenotypes necessary for mapping and cloning the genes responsible for apomixis. This invention uses: plant cyto-embryology procedures to identify and select a plant or group of plants that possess appropriate genetic variability for initiation times and durations of megasporogenesis (female meiosis), embryo sac formation (including egg and central cell maturation), fertilization, embryony and endosperm formation; plant breeding procedures to produce numerous and divergent genetically-recombined progeny; and plant cyto-embryology or progeny test procedures to select segregant plants that express an increased frequency of aposporous or diplosporous embryo sac formation, parthenogenic or adventitious embryo formation, and/or autonomous or pseudogamous endosperm formation.

### BACKGROUND OF THE INVENTION

Apomixis is a natural but rare anomaly that occurs in less than 1% of angiospermous genera (Carman 1997). It does not occur in most of the world's important food and fiber crops, including rice, wheat, maize, barley, millet, sorghum, soybeans, potatoes, most vegetable and oil crops, cotton and many others (Asker and Jerling 1992). It is among such crops that apomixis holds it's greatest potential for providing commercial and humanitarian benefits. Conferring apomixis to world crops could benefit crop production in at least three ways.

First, inbred crops, such as wheat, rice and soybeans, could be converted to superior-yielding hybrid crops such that hybrid vigor is permanently inherited from seed to seed. Today, wheat hybrids yield up to 15% more grain than inbred varieties, but the vast majority of wheat grown internationally is varietal—not hybrid. The high cost of producing hybrid seed, compared to the low cost of producing varietal seed, currently limits the use of hybrid wheat seed to the very highest wheat production areas in the world (Guillen-Portal et al 2002).

It is the anatomy, physiology and genetics of most world crops that currently prevent the economic production of mass quantities of hybrid seed. These economics continue to prohibit a world-wide conversion from inferior varieties to superior hybrids. Apomixis could eliminate this economic bottleneck. For rice, the full exploitation of hybrid vigor could raise rice yields by 30% to 50% over yields of inbred varieties currently grown on the vast majority of rice acreage worldwide (Yuan 1993). By conferring apomixis to hybrids of rice and other major world crops, hybrid seed would be as cheap to produce as varietal seed. This is because apomictic hybrids clone themselves asexually from seed to seed, i.e. from one seed generation to the next. In essence, apomictic seed production systems do not require costly cross-pollination procedures for producing hybrid seed.

Second, apomixis could enhance crop production by reducing costs associated with producing hybrid seed of crops currently grown as hybrids. For example, hybrid seed of corn is produced by identifying genetically-divergent inbred parent lines that when crossed or double-crossed with each other produce superior-yielding hybrid progeny. Once appropriate parent lines are identified or bred, mass cross-pollinations are required to produce commercial quantities of hybrid seed. Apomixis could eliminate most of the cross-pollination costs, i.e. once an apomictic hybrid is produced, it clones itself through it's own seed, generation after generation. Seed companies in the U.S. currently spend about $600 M per year to produce hybrid corn seed. Apomixis could eliminate the cross-pollination procedures and save U.S. corn seed producers more than $300 M annually.

Third, apomixis could be used to transfer biotechnological and productivity advances to marginal farmlands in the developed world and to resource poor farmers in developing nations (Toenniessen 2001). Currently, high costs associated with producing hybrid seed or conferring value-added agbiotech traits to crops prohibit the use of hybrids or value-added traits in resource poor areas of the world. Because apomixis perpetuates such value-added traits (hybridity or agbiotech modifications) from seed to seed, apomixis could become a cost-effective vehicle for delivering these traits to resource-poor farmers in poor nations.

Before the major benefits of apomixis can be realized, methods for inducing apomixis and enhancing its expression in major crops must be developed and perfected. The instant specification provides novel methods for such inductions and enhancements.

### Novelty of the Present Invention

The methods of the instant specification increase the expression of apomixis in plants. These methods are not related to nor are they extensions of approaches currently pursued by other scientists. The following discussion of current approaches is provided so as to clearly identify the critical factors that differentiate the methods of the instant specification from all other approaches currently being pursued.

Approaches currently being explored to confer apomixis to sexual crops by other scientists consist of:
1. crossing a wild apomict with a related sexual species followed by introgressive breeding to transfer the hypothesized “apomixis gene(s)” from the wild apomict to the sexual species
2. inducing a mutation(s) in a sexual species in an attempt to create by mutation the apomixis gene(s) de novo followed by selecting plants that express apomixis
3. mapping and cloning the apomixis gene(s) in natural apomicts and transferring such apomixis gene(s) to crops by transformation
4. constructing the apomixis gene(s) de novo followed by the transfer of such gene(s) to crops by transformation.

Though progress has been made with each of these approaches, none has yet succeeded in converting a sexual species to a commercially-viable apomict (Spielman et al 2003, Estrada-Luna et al 2002, Richards 2003). These four approaches are based on a common belief that apomixis is conferred by one to a very few apomixis gene(s) that evolved through mutation or through epigenetic changes to regulatory genes (Koltunow and Grossniklaus 2003). Hence, it is believed that:
1. the apomixis gene(s) or epigenetic regulatory elements can be introgressed through plant breeding into a sexual species from an apomictic species
2. the apomixis gene(s) can be produced in a sexual plant by mutating a normal gene(s)
3. the epigenetic regulatory elements can be produced in a sexual plant by mutation, hybridization, polyploidization, or other chromosomal aberrations
4. the apomixis gene(s) or epigenetic regulatory elements can be mapped in an apomictic plant, cloned, and transgenically inserted into sexual plants
5. the apomixis gene(s) or epigenetic regulatory elements can be created by biochemically modifying existing gene(s) and then transgenically inserting the modified gene(s) into sexual plants.

The approach taken by the inventor of the instant specification is not based on a belief in one to a very few apomixis genes of mutagenic origin, which can be manipulated or created by conventional or molecular breeding procedures; nor is it based on a belief that apomixis is the result of epigenetic changes in gene regulation. Instead, it is based on a series of novel discoveries, made by the inventor, that place apomixis in the category of genetically regulated traits, which are stabilized by structural heterozygosity at the genome level.

The inventor discovered that extensive genetic variability exists among plants of the same species, genus or family for initiation times and durations of megaspore mother cell (MMC) differentiation, megasporogenesis, embryo sac formation, fertilization, embryony, and endosperm formation (referred to hereinafter as components of the germline development sequence, GDS, or ovule development sequence, ODS) relative to the maturity level of the nongametophytic (sporophytic) tissues of the ovule. This variation has now been characterized in the inventor's lab for several species including Antennaria (FIG. 1-3), Tripsacum (FIG. 4), Sorghum (FIG. 5) and Arabidopsis (FIG. 6). The inventor further discovered that this variability is ecotypically partitioned in nature and is controlled by multiple genes with multiple alleles dispersed among related ecotypes of the same species, genus or family. The inventor then hypothesized that the ecotypically-partitioned alleles confer fitness to ecotypes in their respective habitats and that apomictic plants can be produced from sexual plants by (a) hybridizing ecotypes divergent in initiation times and durations of megasporogenesis and embryo sac development, (b) obtaining progeny, and (c) screening for apomixis among the progeny. In the hybrids, competition among asynchronously-expressed developmental signals arises during ovule development, and apomixis occurs, i.e. embryo sac formation preempts megasporogenesis and embryony preempts fertilization (FIG. 7-9; J. G. Carman, 1997, Methods for producing apomictic plants, WO 98/33374, incorporated herein by reference, hereinafter, “WO 98/33374). The inventor then provided methods that can be used to genetically-stabilize apomixis, i.e. to prevent genetic recombination from occurring at the various loci critical to the expression of apomixis (J. G. Carman, 1999, Methods for stabilizing and controlling apomixis, WO 01/32001, hereby incorporated by reference, hereinafter, “WO 01/32001). WO 98/33374 and WO 01/32001 are the result of the inventor:
1. teaching that extensive genetic variability for initiation times and durations of GDS components exists among related plants and that this variation permits apomictic plants to evolve in nature (Carman 1997, 2000, 2001)
2. verifying the existence of genetic variability for initiation times and durations of GDS components (FIG. 1-6)
3. using the genetic variability for initiation times and durations of GDS components to produce apomictic F₁ hybrid plants from sexual plants (FIG. 7-9; WO 98/33374)
4. genetically stabilizing a synthetic apomict by modifying its genomic structure such that sexual reproduction was essentially eliminated (WO 01/32001).

The instant specification describes an important advancement to the inventor's state-of-the-art methods for predictably producing apomictic plants from sexual plants. Before describing this advancement in detail, it is important to note that the inventor's methods (WO 98/33374, WO 01/32001) are the only methods published to date for predictably producing apomictic plants from sexual plants and genetically stabilizing them. In this respect, WO 98/33374 and WO 01/32001 teach that apomixis can be induced by creating F₁ hybrids in which developmentally-disruptive competition occurs between asynchronously-expressed developmental programs (FIG. 10). When parent lines are appropriately chosen, the expression of both megasporogenesis and embryo sac formation signals are temporally superimposed in ovules of F₁ hybrids. Embryo sac formation in such hybrids often tends to preempt megasporogenesis or nucellar cell development, which causes diplosporous or aposporous embryo sacs to form, respectively (FIG. 7-9).

It is further taught in WO 01/32001 that sexually-derived progeny from a “genetically-unstable apomict” usually will have largely reverted to sexuality due to genetic recombination at the multiple loci responsible for apomixis. Genetic segregation during facultative sexual reproduction in a genetically-unstable facultative apomict tends to produce progeny in which the allelic combinations that cause the asynchronous competition responsible for apomixis are disrupted. Accordingly, sexually-derived progeny of a genetically-unstable facultative apomict tend to express less penetrance for apomixis. WO 01/32001. In contrast, Applicant surprisingly discovered that a low percentage of sexually-derived progeny of certain genetically-unstable facultative apomicts will actually express a higher level of apomixis due to specific and infrequent recombination events the frequency of which can be predicted by plant breeders. In this context, the inventor has discovered that genes regulating initiation times and durations of:
1. MMC differentiation are largely independent (barring linkage) of genes that regulate initiation times and durations of megasporogenesis, embryo sac formation, fertilization, embryony, and endosperm formation
2. megasporogenesis are largely independent (barring linkage) of genes that regulate initiation times and durations of embryo sac formation, fertilization, embryony, and endosperm formation
3. embryo sac formation are largely independent (barring linkage) of genes that regulate initiation times and durations of fertilization, embryony, and endosperm formation
4. fertilization are largely independent (barring linkage) of genes that regulate initiation times and durations of embryony and endosperm formation
5. embryony are largely independent (barring linkage) of genes that regulate initiation times and durations of endosperm formation (FIG. 1-6).

The significance of this discovery is that initiation times and durations of the various GDS components, and even processes within each GDS component, are not controlled exclusively by the same genes. Based on these discoveries, the inventor theorized that:
1. initiation times and durations of GDS components can be genetically uncoupled from each other through plant breeding and selection
2. lines that express higher levels of apomixis (more ovules per plant expressing apomixis) can be produced by intentionally modifying, through plant breeding or molecular procedures, initiation times and durations of certain GDS components
3. sexually-recombinant progeny of a man-made or natural genetically-unstable apomict will express higher levels of apomixis if GDS components are temporally uncoupled such that signals for early megasporogenesis are lost while signals for early embryo sac formation are retained and/or signals for early egg cell formation and fertilization are lost while signals for early embryony and endosperm formation are retained. Data presented herein indicate that onset timings of GDS stages, such as meiosis and embryo sac formation, are quantitatively inherited traits such that transgressive segregation (Rieseberg et al, 2003) for these traits can be expected among segregating progeny as is reported herein (FIG. 11).
4. pairs of plants exhibiting allelic variability for embryological phenotypes necessary to produce apomictic plants can also be used to map and clone genes responsible for apomixis.

Starting with a genetically-unstable facultative apomict, the percentage of sexually-derived progeny (genetically recombined) that express an enhanced level of apomixis will generally be low, but within the scope of screening procedures common to the plant breeding industry. This novel approach for increasing the frequency of apomixis expression in angiosperms has been tested and reduced to practice (FIG. 11-14), and patent protection is hereby requested.

The instant specification discloses methods that increase the frequency of apomixis expression in angiosperms. These newly disclosed methods were made possible by the inventor's surprising discoveries concerning the genetic control of initiation times and durations of GDS components. The inventor discovered that a low percentage of sexually-derived progeny of certain genetically-unstable facultative apomicts will actually express a higher level of apomixis due to specific and infrequent recombination events. Further study and development based on this surprising discovery lead to the presently disclosed methods of producing and/or enhancing apomicitic plants.

### SUMMARY OF THE INVENTION

It is an object of the instant specification to provide methods for producing apomictic plants from sexual angiospermous plants or from angiospermous plants that express a lower frequency of apomixis expression than the apomictic plants produced. Another object of the instant specification is to provide new methods for producing apomixis-enhanced plants that express a higher frequency of one or more of the various components (or elements) of apomixis relative to the plant materials from which said apomixis-enhanced plants were produced. Elements of apomixis include unreduced embryo sac formation (aposporous or diplosporous), parthenogenesis or adventitious embryony, and autonomous or pseudogamous endosperm formation. A further object of the instant specification is to provide novel methods for enhancing genetic variability within individual plants for initiation times and durations of various GDS components including megasporogenesis, embryo sac formation (including egg and central cell formation and maturation), fertilization, embryo formation and endosperm formation.

Additional objects and advantages of the present invention are set forth in the detailed description or will be appreciated by the practice of the invention.

To address the foregoing objects, and in accordance with the invention as described herein, the instant specification provides methods for:
producing an apomictic plant from starting plant materials consisting of:
   sexual plants;
   facultatively-apomictic plants that are less apomictic than said apomictic plant; or
   sexual plants and facultatively-apomictic plants, which are less apomictic than said apomictic plant.
producing an apomictically-enhanced plant that expresses a higher frequency of one or more of the various elements of apomixis relative to starting plant materials that may consist of either sexual or facultatively-apomictic plants;
selecting starting plant materials from which said apomictic or apomictically-enhanced plants are produced; and
identifying said apomictic or apomictically-enhanced plants from among putative apomictic or apomictically-enhanced plants.
   Specifically, the starting plant materials may consist of:
a sexual plant;
a facultatively-apomictic plant that expresses a lower frequency of either apomixis or an element thereof than said apomictic or apomictically-enhanced plant;
two or more sexual plants of the same or related species (within the same family);
two or more facultatively-apomictic plants of the same or related species each of which expresses a lower frequency of either apomixis or an element thereof than said apomictic or apomictically-enhanced plant; or
two or more plants of the same or related species one or more of which expresses only sexual reproduction and one or more of which expresses a lower frequency of either apomixis or an element thereof than said apomictic or apomictically-enhanced plant.

It will be appreciated that numerous plants may be produced by the methods of the instant specification, and that the numerous plants produced will range in apomixis expression from less expression to more expression relative to the starting plant materials.

In one embodiment, the present invention is directed to a method of producing an apomictic plant having a frequency apomictic seed set exceeding that of the parent plant from which the apomictic plant was produced. The invention is also directed to a method of producing a plant that expresses an increased frequency of one or more of the various elements of apomixis. The elements of apomixis preferably include unreduced embryo sac formation (aposporous or diplosporous), parthenogenesis or adventitious embryony, and autonomous or pseudogamous endosperm formation. Generally, these methods include the steps of: (a) obtaining a parent plant that expresses one or more elements of apomixis and is not genetically stable for the elements of apomixis; (b) self fertilizing the parent plant or sib-mating the parent plant with another related parent plant that also expresses elements of apomixis, but is not genetically stable for the elements of apomixis; (c) obtaining seed from the parent plants; (d) sowing the seed obtained; (e) raising progeny plants there from; (f) screening the progeny plants for an increased frequency of apomictic seed set as compared to the parent plants; and (g) isolating the progeny plant expressing the increased frequency of apomictic seed set.

Preferably the frequency of apomictic seed set in the isolated progeny plant produced is at least 5% greater than the parent plants, more preferably at least 20% greater than the parent plants, and most preferably 40% greater than the parent plants.

In this embodiment, steps (b) through (e) can optionally be repeated at least one time to obtain second generation or higher generation progeny having an increased frequency of apomictic seed set compared to the previous generation.

Preferably the sib-mating is a full sib-mating or a half sib-mating, but other broad sib-matings are envisioned and intended to be within the scope of the invention.

In yet another embodiment, the parent plant is obtained by:
identifying ecotypes or breeding lines from the breeding population that represent extremes in GDS timing;
selecting from an ecotype or breeding line that represents extremes in GDS timing a first and second plant, wherein the mean onset time for embryo sac formation of the first plant occurs shortly after or before the mean onset time for megasporogenesis of the second plant relative to the maturity level of sporophytic ovule or ovary tissues;
hybridizing the first and second parent plants;
obtaining seed from the first or second plants;
sowing the seed obtained;
raising progeny plants there from; and
identifying plants that expresses elements of apomixis and are not genetically stable for the elements of apomixis to obtain the parent plant.

The invention is also directed to a method for selecting a group of plants to be used as a breeding population for producing plants that express apomixis. This method preferably includes the following steps:
(a) selecting genetically-divergent ecotypes or breeding lines of the same angiospermous species, genus or family;
(b) GDS-characterizing said ecotypes or breeding lines relative to the maturity level of nongametopytic ovule or ovary structures;
(c) including in the breeding population ecotypes or breeding lines that represent extremes and midpoints in GDS timing such that plants are included in which:
   GDS stages (megasporogenesis, embryo sac formation, fertilization, embryony and endosperm formation) in a plant occur early relative to the maturity level of sporophytic ovule and ovary structures,
   GDS stages in a plant occur late relative to the maturity level of sporophytic ovule and ovary structures,
   some GDS stages in a plant occur early while others occur late relative to the maturity level of sporophytic ovule or ovary structures.

The present invention is further directed to a method for selecting parent plants from a breeding population to produce plants that express an increased frequency of apomictic embryo sac formation and to a method of selecting parent plants from a breeding population for the purpose of producing plants that express an increased frequency of parthenogenesis, adventitious embryony, pseudogamous endosperm formation, or autonomous endosperm formation. These methods preferably include the steps of identifying ecotypes or breeding lines from the breeding population that represent extremes in GDS timing; and selecting from the identified ecotypes or breeding lines pairs of plants to be used as parents such that the mean onset time for embryo sac formation in one parent occurs early relative to the maturity level of sporophytic ovule or ovary tissues while the mean onset time for female meiosis (megasporogenesis) in the other parent occurs late.

The present invention also encompasses a method of producing an apomictic or apomictic-enhanced progeny plant from sexual or facultatively-apomictic parent plants comprising the steps of:

(a) selecting a first and second sexual or facultatively apomictic parent plant from an angiospermous plant species, genus, or family, wherein the mean onset time for embryo sac formation in the first parent plant occurs at about the same time as or before the mean onset time for megasporogensis in the second parent plant relative to the maturity level of sporophytic ovule or ovary tissue;

(b) hybridizing the first and second parent plants;

(c) obtaining seed from the first or second plants;

(d) sowing the seed obtained;

(e) raising progeny plants there from;

(f) identifying progeny plants that expresses elements of apomixis and is not genetically stable for the elements of apomixis;

(g) self-fertilizing or sib-mating one or more progeny plants identified;

(h) obtaining second generation seed from the progeny plants;

(i) sowing the obtained second generation seed obtained;

(j) raising second generation plants there from; and

(k) screening and identifying the second generation plants that are apomictic.

In yet another embodiment of the invention, the invention is a method of producing an apomictic progeny plant from sexual or facultatively-apomictic parent plants comprising the steps of:

(a) selecting genetically-divergent ecotypes or breeding lines of the same angiospermous species, genus or family;

(b) characterizing the ecotypes or breeding lines according to germline development sequence (GDS) relative to the maturity level of sporophytic ovule or ovary structures;

(c) producing a breeding population that includes ecotypes or breeding lines that represent extremes in GDS timing comprising:
plants having GDS stages that occur early relative to the maturity level of sporophytic ovule and ovary structures and plants having GDS stages that occur late relative to the maturity level of sporophytic ovule and ovary structures; or
plants having GDS stages that occur early while others occur late relative to the maturity level of sporophytic ovule or ovary structures;

(d) identifying ecotypes or breeding lines from the breeding population that represent extremes in GDS timing;

(e) selecting parent plants from the identified ecotypes or breeding lines, wherein the selected parent plants have:
a mean onset time for embryo sac formation in one parent plant that occurs shortly after or before the mean onset time for megasporogenesis in the other parent plant relative to the maturity level of sporophytic ovule or ovary tissues; and
mean onset times for embryo and endosperm formation in one parent plant that occur shortly after or before the mean onset times for the mature embryo sac, maturing egg, maturing central cell and fertilization stages in the other parent plant relative to the maturity level of sporophytic ovule or ovary tissues;

(f) crossing the parent plants, obtaining seed there from, sowing the seed, raising F₁ progeny plants, self fertilizing or intercrossing the F₁ progeny, obtaining F₂ or double cross seed from the F₁ plants, sowing the F₂ or double cross seed, raising the F₂ or double cross progeny there from; and

(g) screening F₂ or double cross progeny for an increased frequency of apomictic seed set as compared to the parent plants.

In this embodiment, step (f) can optionally be repeated at least once to obtain advanced breeding generations, followed by screening the advanced generation plants for an increased frequency of apomictic seed set as compared to the parent plants.

The methods of the invention can also further comprise the step of doubling the chromosome number of the progeny to stabilize apomixis. The invention further is directed to apomictic or apomictic-enhanced plants produced according to the methods disclosed herein, and progeny thereof.

Preferably, the plant used in the methods of the invention is a rice, wheat, maize, barley, sorghum, millet, soybean, potato or cotton plant. In a preferred embodiment, the plant is sorghum.

### BRIEF DESCRIPTION OF THE FIGURES

FIG. 1. Representative DIC photomicrographs of the dyad and ES-2 stages from specific accessions of A. corymbosa, A. aromatica, A. umbrinella, A. marginata and A. microphylla. The long double-arrowed lines in the photomicrographs depict ovule length, which consists of the funiculus length plus the length of chalazal tissue distil to the funiculus. Integument lengths in the photomicrographs are depicted by the shorter double-arrowed lines and include the length of the distal integument structure plus the length of the proximal chalazal tissue. The latter gives rise to the integument. c=chalaza, d=dyad, f=funiculus, i=integument, n=nucellus. (Kowallis, Carman and Bayer, in preparation)

In angiosperms, the nucellus and MMC begin to differentiate early in development of the funiculus and chalaza, and prior to integument differentiation. Differentiation of the integument(s) then initiates by periclinal divisions in the epidermis of the chalaza. The chalaza, funiculus and integument(s) remain largely undifferentiated during megasporogenesis and early embryo sac formation (Esau 1977). In Antennaria, zones of coordinated periclinal cell divisions in intercalary meristematic tissues located near the chalaza/funiculus and chalaza/integument junctions cause the ovule to curve and assume an anatropous form (see photomicrographs, left column). Technically, the integument does not grow around the developing gametophyte, as is often stated in the literature. Instead, the terminus of the integument remains close to the funiculus base, and elongation of intercalary-meristem-produced cells causes the chalaza and chalaza-attached meiocyte or gametophyte to recede deep into the ovarian cavity (locule).

The formation of anatropous ovules was observed to be a canalized process that occurred essentially identically for all Antennaria species evaluated. In this respect, the distance from the integument terminus to the funiculus origin was similar for all species studied and did not change appreciably during embryo sac development (FIG. 2). Across species studied, cell divisions, cell growth and cell differentiation in the funiculus, chalaza and integument were highly coordinated processes that resulted in anatropous ovules of essentially identical shape.

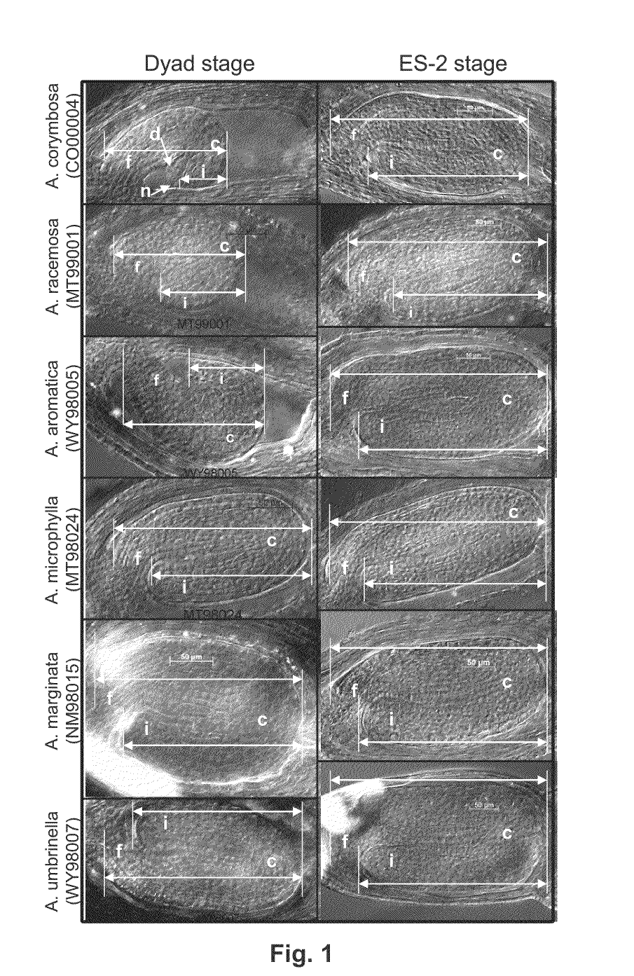

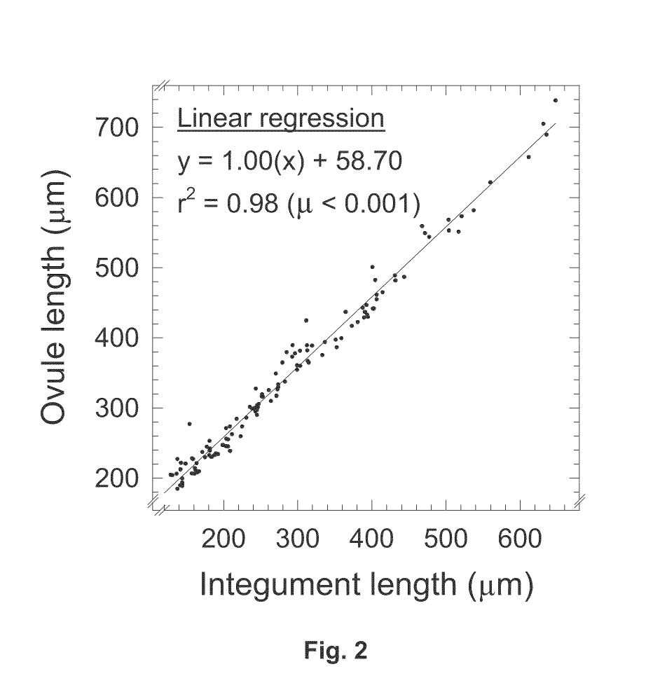

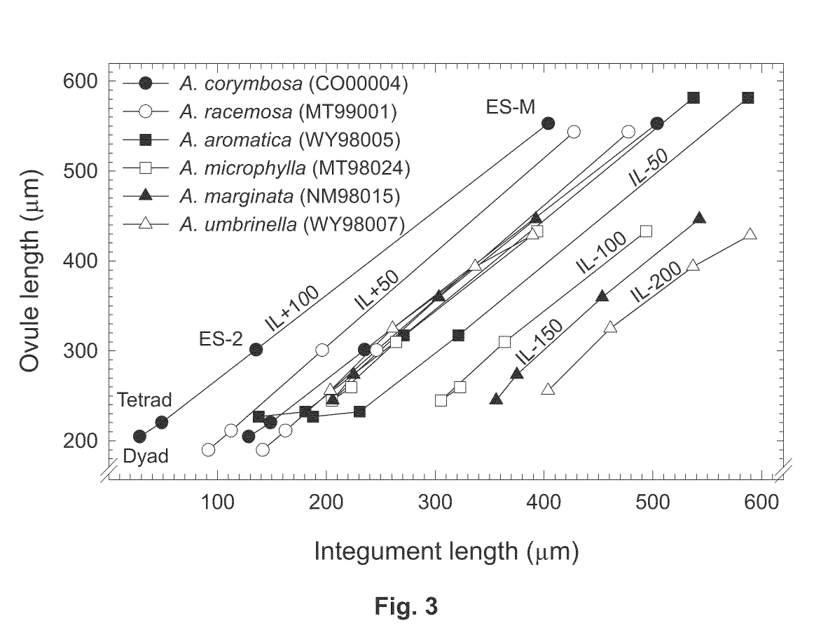

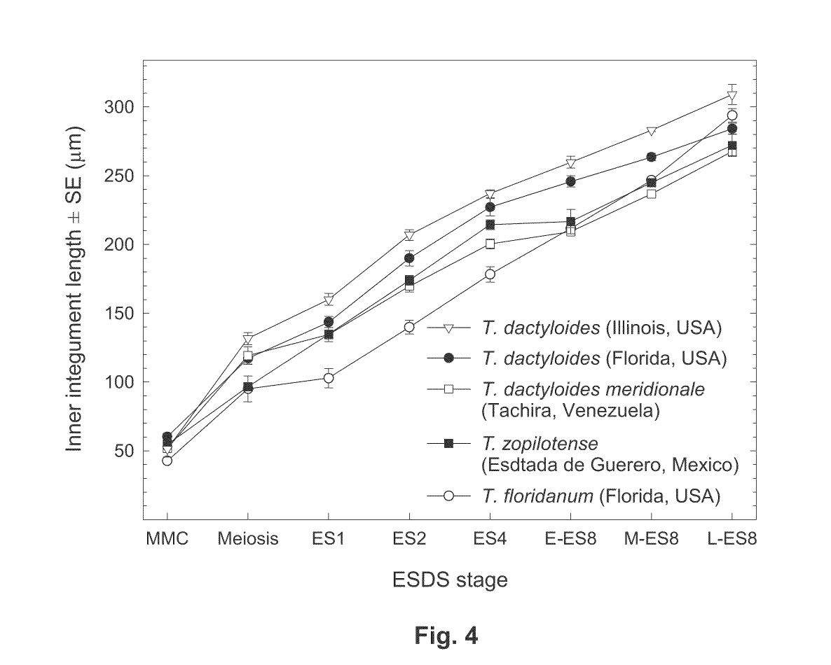

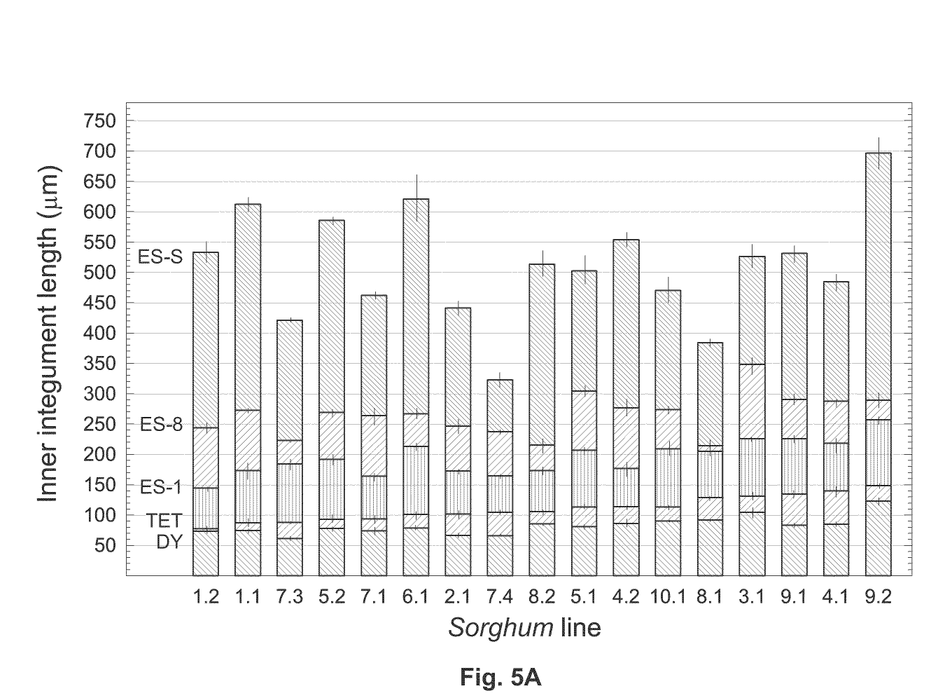

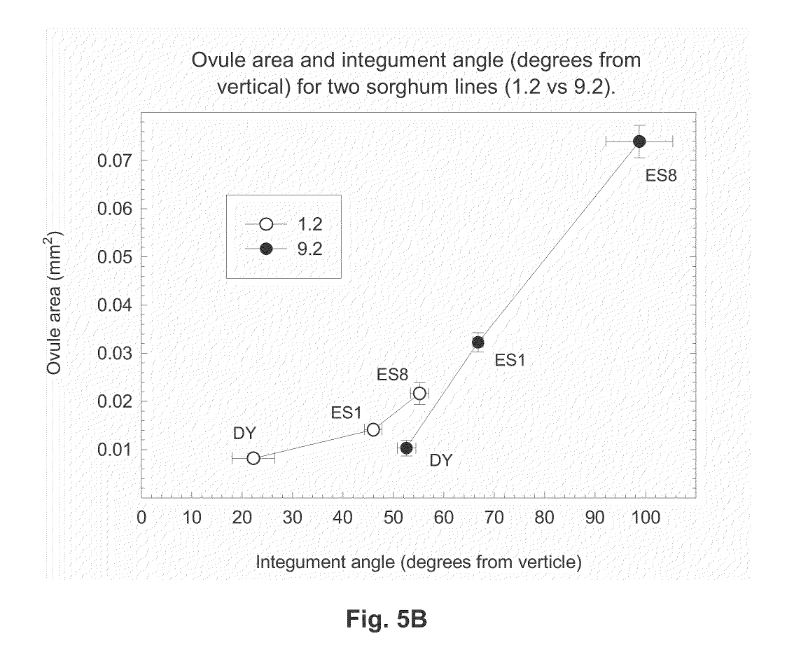

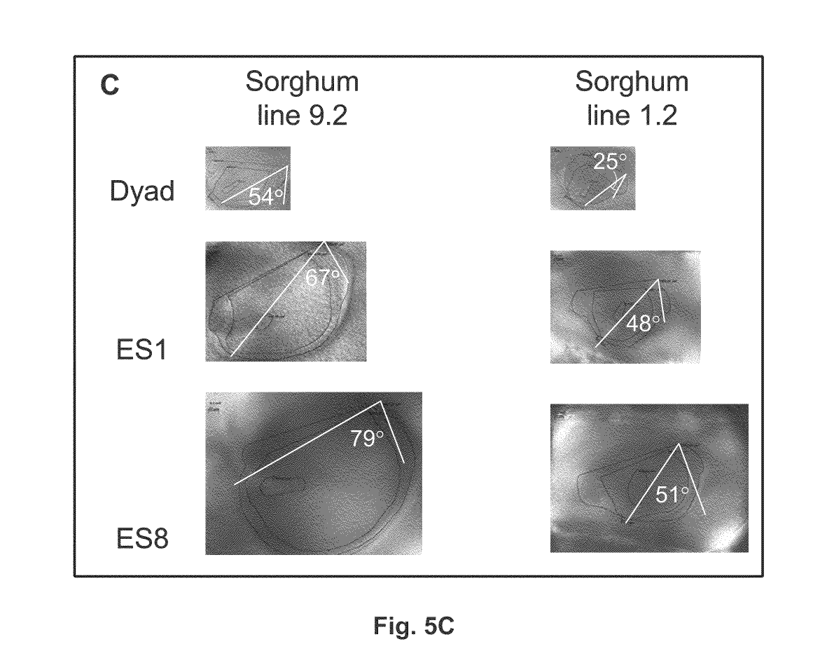

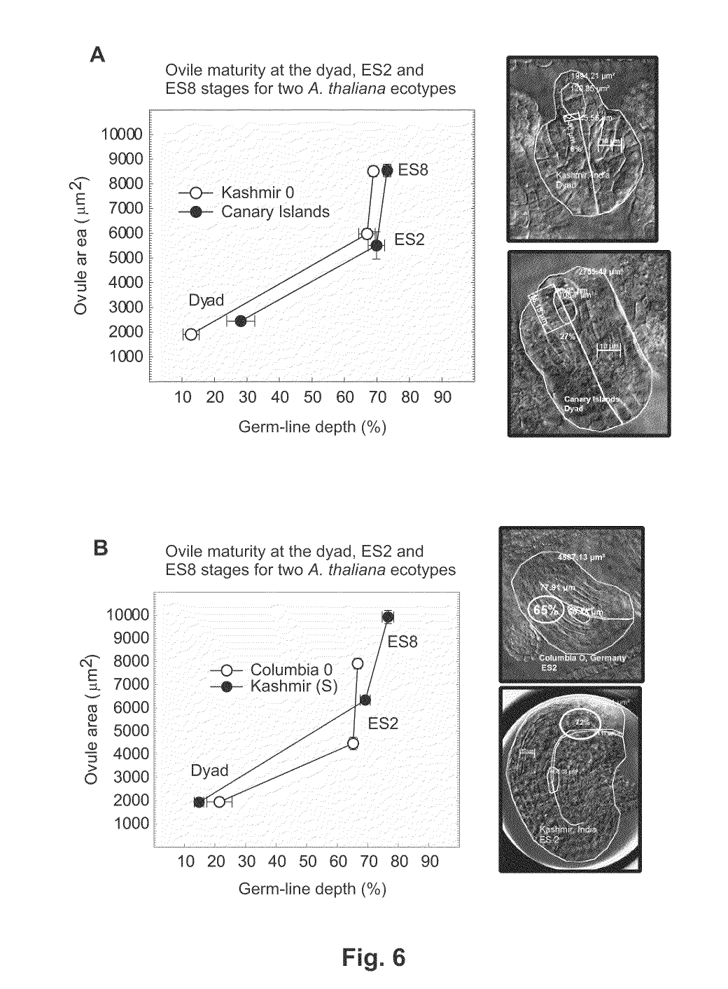

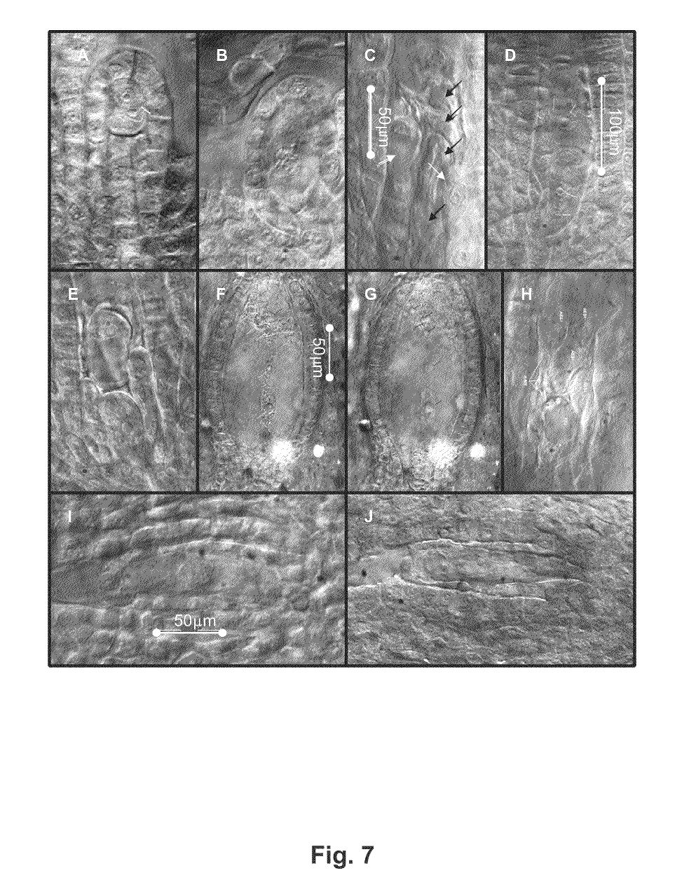

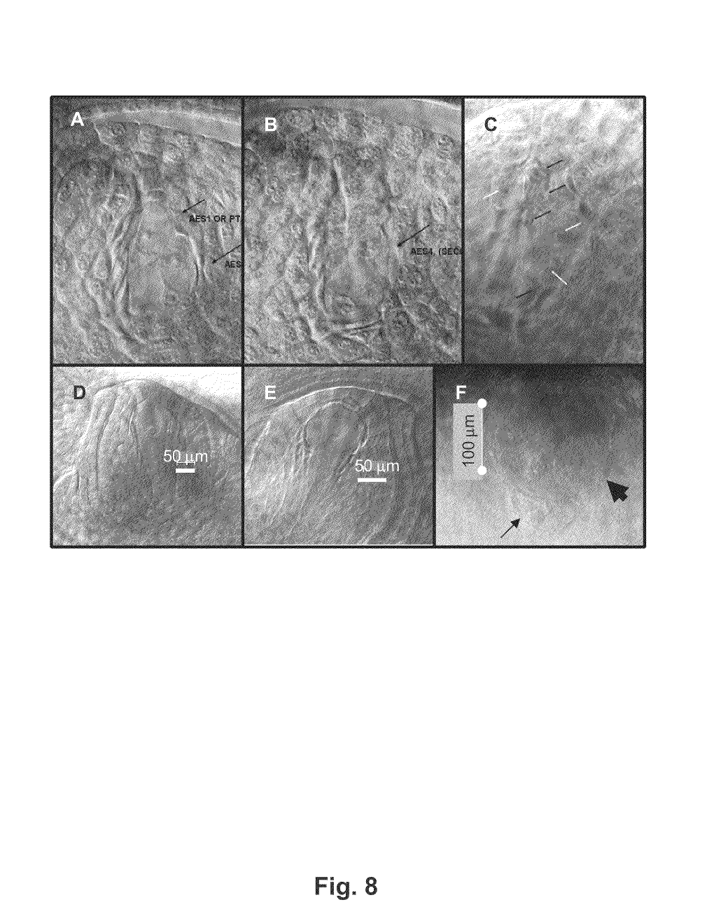

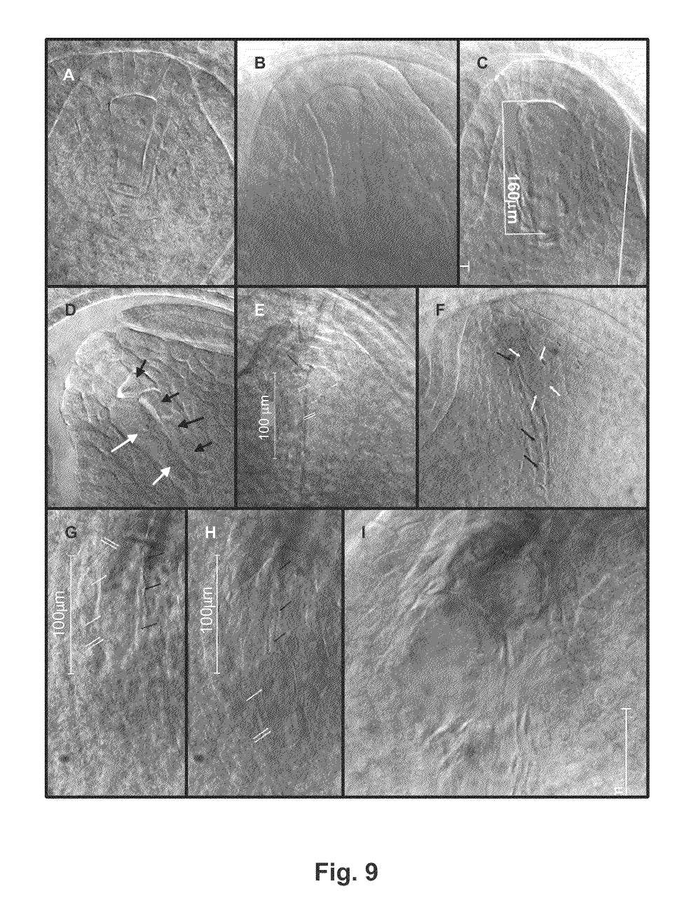

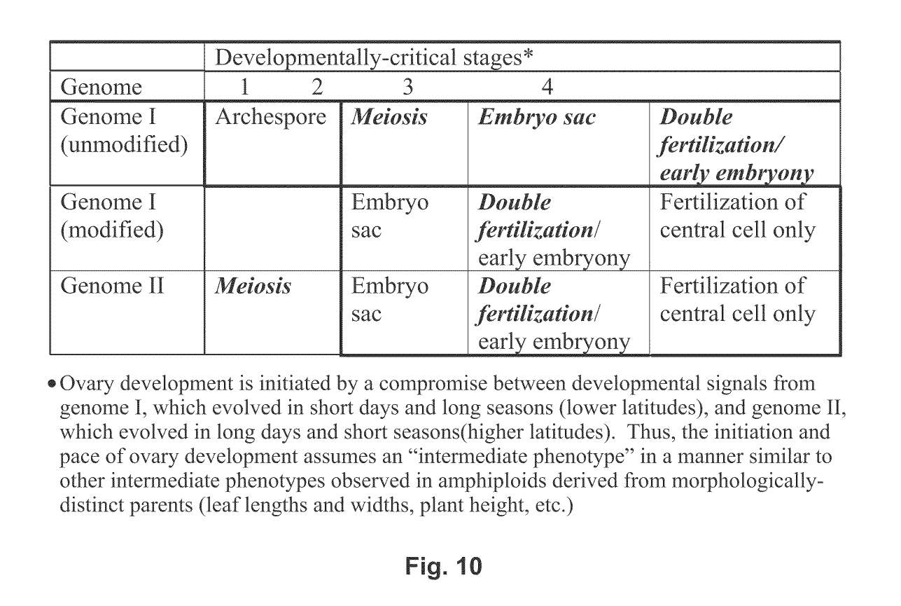

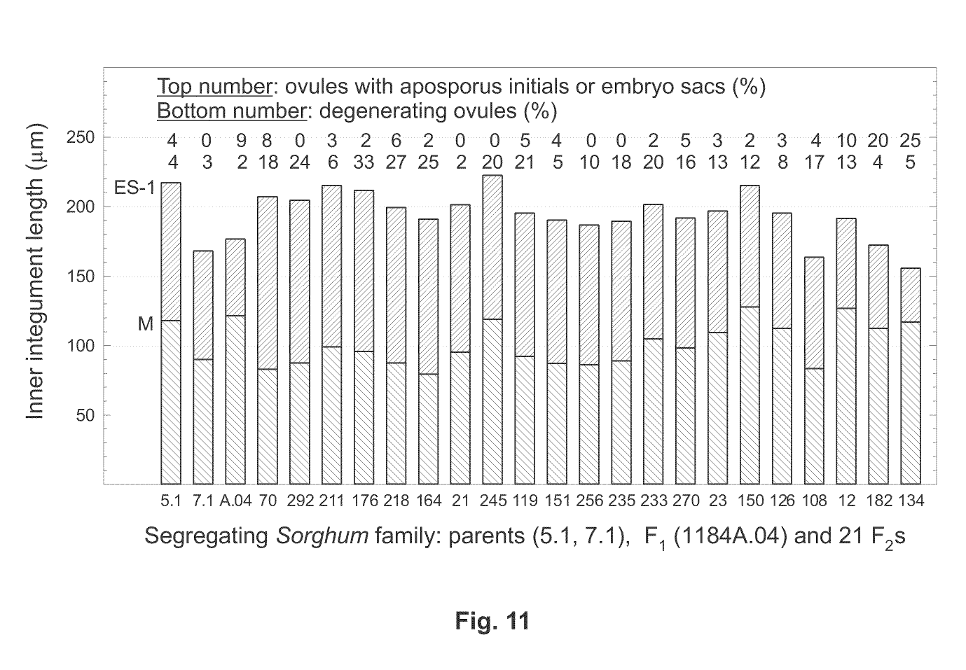

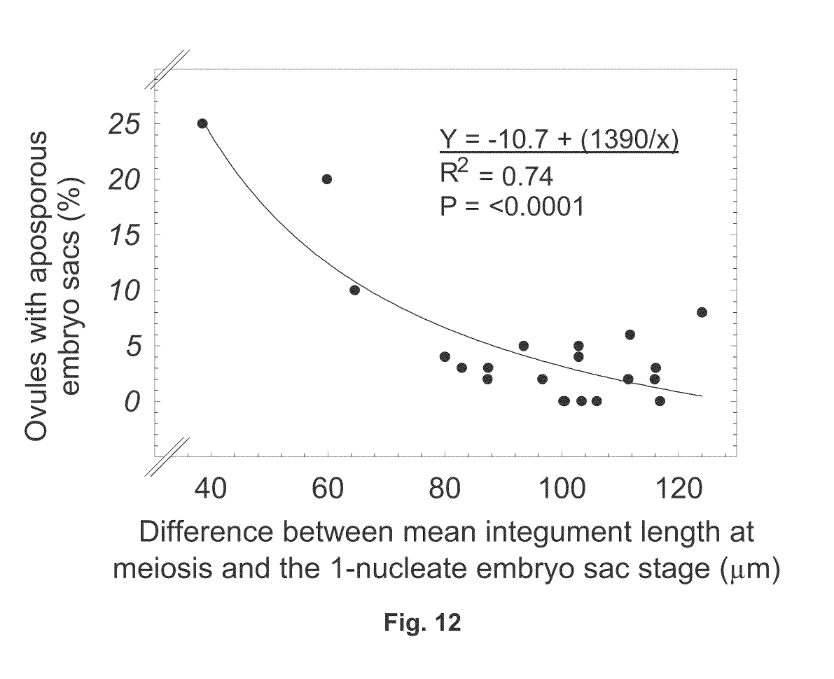

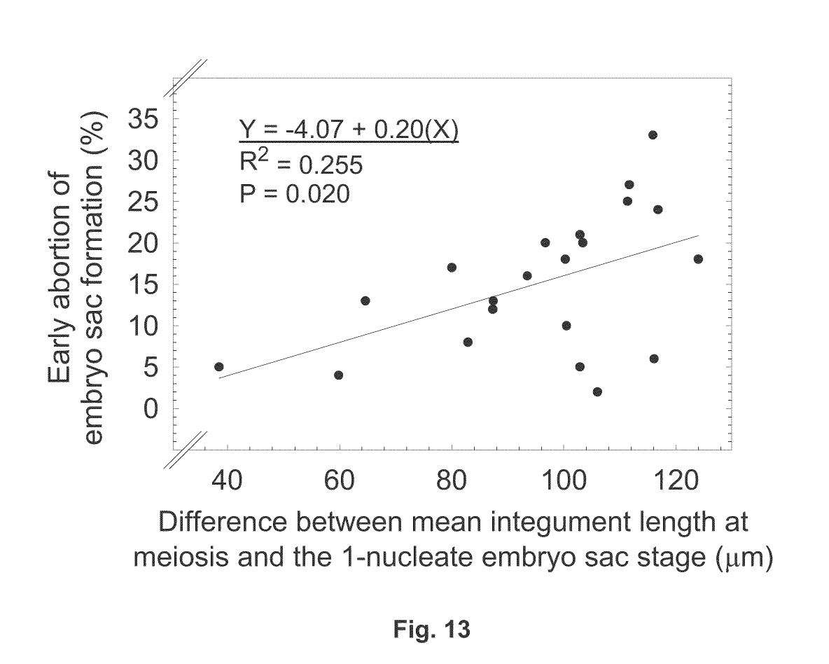

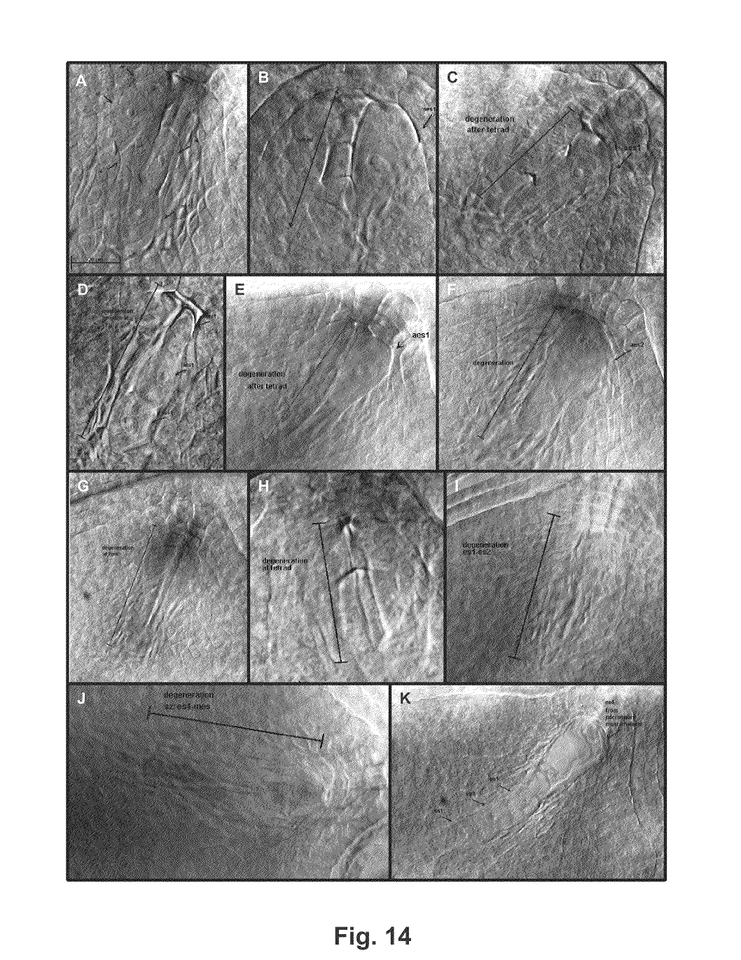

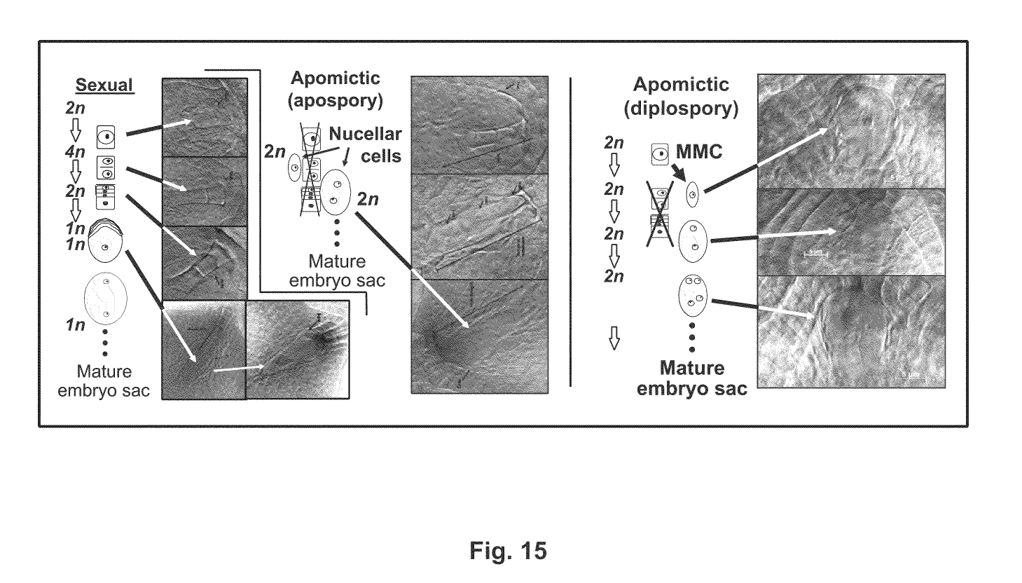

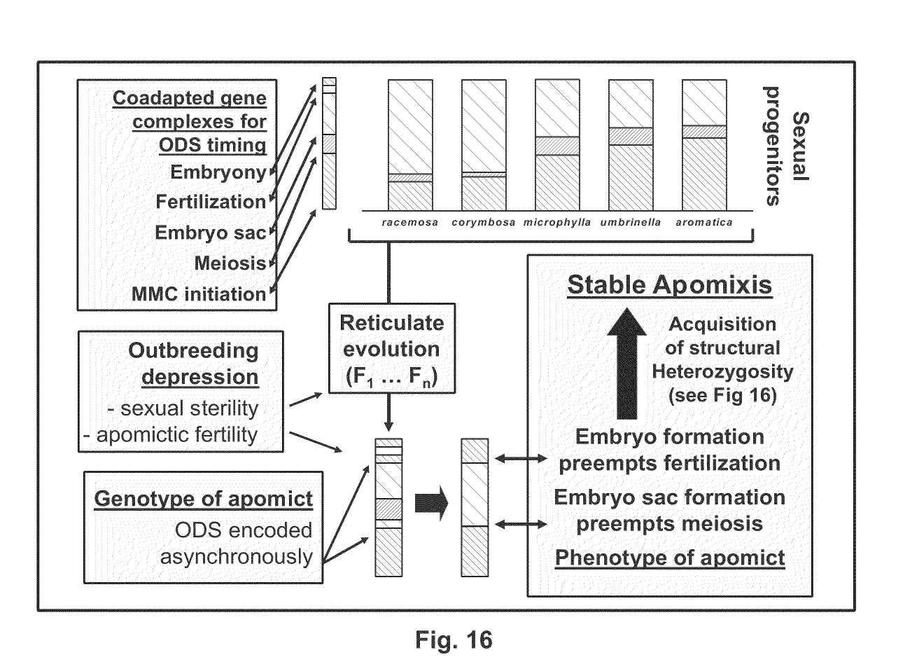

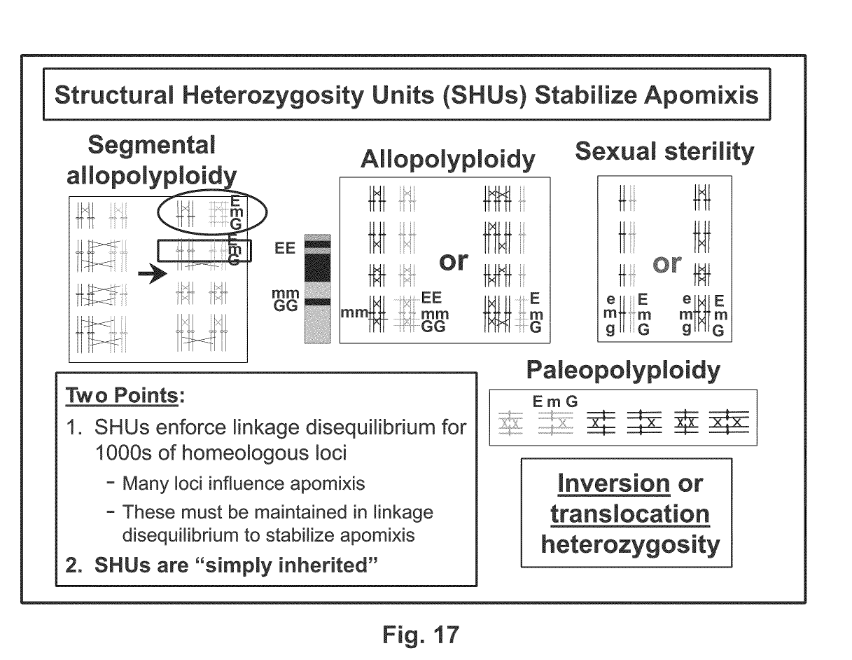

Anatropous ovule formation appears to be developmentally canalized across the Antennaria species studied. In contrast, much genetically-determined heterochronicity was observed among accessions (FIG. 3) for initiation times and durations of megasporogenesis and embryo sac development relative to the changing size and shape of the anatropous ovule. The dyad stage was often reached prior to full achievement of the anatropous form in A. corymbosa, A. racemosa and A. aromatica but well after achievement of this form in A. microphylla, A. marginata and A. umbrinella (compare FIG. 1 photomicrographs of dyads with FIG. 2-3).

FIG. 2. Linear regression of ovule lengths vs integument lengths at the dyad, tetrad, 2-nucleate embryo sac and mature embryo sac stages for 31 Antennaria accessions. Among accessions studied, ovule growth rates tended to equal integument growth rates (y=1.00(x)), and the distance from the integument terminus to the base of the funiculus (FIG. 1) varied little from 58.7 Φm. Data from A. aromatica (MT00005, MT98034, MT98043, WY98005), A. racemosa (MT00003, MT00004, MT00006, MT99001), A. corymbosa (CO00001, CO00004, MT00001, MT99006), A. umbrinella (CO98031, MT98026, MT98042, MT99003, WY98007, WY99004), A. microphylla (CO98001, CO98006, CO98009, CO98037, CO98029, MT98001, MT98007, MT98024, WY98003), A. marginata (NM98015), A. densifolia (YK98006, YK98007), and A. rosulata AZ98008) were included. Accessions were collected and accession numbers were assigned by the inventor. (Kowallis, Carman and Bayer, in preparation)

FIG. 3. Mean ovule and integument lengths at four GDS stages for six accessions of Antennaria sp. collected from New Mexico, Colorado, Wyoming and Montana. The group of six parallel, superimposed lines in the middle of the figure represent the observed mean ovule and integument length values for the six accessions depicted in FIG. 1 (these data also contributed to the regression analysis, FIG. 2). The six non-superimposed lines are the same as those in the figure, but integument length values for each line were adjusted to facilitate discussions of heterochronic differences among lines in initiation times and durations of various GDS stages relative to the maturity level of the sporophytic anatropous ovule tissues. The four points on each line, starting at the lowest and moving upward, represent the dyad, tetrad, 2-nucleate embryo sac (ES-2) and mature embryo sac (ES-M) stage, respectively. The label “IL+100”, associated with A. corymbosa accession CO00004, means that a value of 100 should be added to the integument length value at each point on the line to arrive at the observed integument length values (as found in the center of the figure). Similar adjustments were made to the remaining lines so as to spread the lines apart.

The unadjusted lines in FIG. 3 are parallel and superimposed, which indicates that growth of the chalaza, funiculus and integument of the anatropous ovule are developmentally-canalized events, i.e. development is largely invariable among accessions (compare with FIGS. 1, 2). In contrast, extensive genetic variation was observed among accessions for initiation times and durations of GDS stages relative to the maturity level of the anatropous ovule. For example, the dyad and tetrad stages in the A. corymbosa, A. racemosa and A. aromatica accessions occur on average while the ovule is still small and immature. In contrast, the dyad and tetrad stages in the A. umbrinella, A. marginata and A. microphylla accessions occur when the ovule is larger and more mature (compare with FIG. 1). Note that the ES-2 stage in A. marginata and A. umbrinella occurs when the integument/funiculus structure is much more mature. Note also that the timing of the dyad and tetrad stages is not correlate with the timing of the ES-2 and ES-M stages. This is strong evidence that the initiation time for each GDS stage is at least somewhat independent of the initiation times for other GDS stages, i.e. initiation times and durations of GDS stages are at least somewhat genetically independent of each other. (Kowallis, Carman and Bayer, in preparation)

FIG. 4. Mean inner integument length at eight GDS stages for five accessions of Tripsacum sp. originating from Illinois, Florida, Venezuela and Mexico. Extensive genetic variation was observed among accessions for initiation times and durations of GDS stages relative to the maturity level (length) of the inner integument. The dyad and tetrad stages in T. zopilotense and T. floridanum occurred on average while the inner integument was still small and immature. In contrast, the dyad and tetrad stages in the T. dactyloides and T. dactyloides var meridionale occurred when the inner integument was much larger and more mature. Note that the ES-1 stage in T. floridanum occurred, on average, before meiosis occurred in the T. dactyloides accessions. Note also that the timing of meiosis, etc, is not correlate with the timing of other GDS stages. This is strong evidence that the initiation time for each GDS stage is somewhat independent of the initiation time for other GDS stages, i.e. initiation times and durations of GDS stages are somewhat genetically independent of each other. Identity of Tripsacum lines: T. dactyloides, Illinois, WW-1276 (USDA, Woodward, Okla.); T. dactyloides, Florida, WW-2435; T. dactyloides var meridionale, MIA 34575 (USDA, Miami), Santander, Columbia; T. zopilotense, 7129-4 (CIMMYT, Mexico), Cañon del Zopilote, Esdtado de Guerero, Mexico; T. floridanum, Florida, MIA 34719. (Bradley and Carman, in preparation)

FIG. 5. Different ways of measuring GDS variation among Sorghum bicolor lines. (A) Mean inner integument length at five GDS stages for 17 accessions (DY=dyad stage, TET=tetrad stage, ES-1=1-nucleate embryo sac stage, ES-8=early 8-nucleate embryo sac stage, ES-S=stigma exertion stage). Extensive genetic variation was observed among accessions for initiation times and durations of GDS stages relative to the maturity level (length) of the inner integument. Note that the initiation of embryo sac formation (bottom of yellow bar) in accessions 1.2, 1.1, 7.3, 5.2 and 7.1 occurred, on average, before the dyad stage of meiosis (bottom of orange bar) was reached in accessions 3.1 and 9.2. Note also that the timing of the dyad and tetrad stages is not correlate with the timing of the ES-1, ES-8 and ES-S (embryo sac at stigma exertion) stages. This is strong evidence that initiation timing for each GDS stage is largely independent of initiation timing for other GDS stages, i.e. initiation times and durations of GDS stages are largely genetically independent of each other. Identity of Sorghum lines: 1.2, Sorghum bicolor, Aispuri (converted), PI 533817 (USDA, GRIN); 1.1, Sorghum bicolor, Aispuri, PI 253638; 7.3, Sorghum bicolor (hybrid), 0-756, PI 302166; 5.2, Sorghum bicolor, Westland, NSL 4003 (USDA); 7.1, Sorghum halepense, 1111, PI 542649; 6.1, Sorghum bicolor, Colby, PI 571105; 2.1, Sorghum bicolor, Kafir (IS 2942), NSL 51477; 7.4, Sorghum halepense, Zhuronskiya, PI 539065; 8.2, Sorghum arundinaceum, R-319, PI 329251; 5.1, Sorghum bicolor, Early Kalo, NSL 3999; 4.2, Sorghum bicolor, Karad Local, PI 248318; 10.1, Sorghum bicolor, Lydenberg Red, IS 2386 (ICRISAT); 8.1, Sorghum arundinaceum, IS 12693, PI 225905; 3.1, Sorghum bicolor, IS 3922, NSL 51483; 9.1, Sorghum bicolor, Vir-5049, PI 562347; 4.1, Sorghum bicolor, Karad Local, PI 533932; 9.2, Sorghum bicolor, Agira, PI 217855. (B) Ovule areas and integument angles (degrees from verticle, see FIG. 5C) for two GDS-divergent sorghum lines at the DY, ES1 and ES8 stages. Note that the ES1 stage occurs at a smaller integument angle (less mature ovule) in line 1.2 on average than the DY stage in line 9.2 (see FIG. 5C). (C) Photomicrographs of the dyad, ES1 and ES8 stages showing differences in ovule size (all photomicrographs are at the same magnification) and integument angle (see FIG. 5B) between lines 9.2 and 1.2. Note that line 1.2 achieves megasporogenesis and embryo sac formation much earlier in ovule development (size and integument angle) than line 9.2 (see FIG. 5A,B).

FIG. 6. GDS variation among Arabidopsis thaliana ecotypes; ES2=pre-vacuolate 2-nucleate embryo sac stage, ES8=early 8-nucleate embryo sac stage. Top graph and figures: mean ovule area and depth of the germ-line structure (dyad, ES2 or ES8) for two ecotypes (Kashmir O and Canary Islands). Ovule area is taken from sagittal sections and includes chalaza, integuments and nucellus. The depth of the germ-line structure is determined by drawing a line from the chalazal perimeter of the area measurement to the tip of the integuments and then determining how deep the chalazal end of the germ-line structure is relative to this line (see photomicrograph inserts). Note that the dyad stage occurs much earlier in development (percentage germ-line depth) in Kashmire O than in the Canary Islands line (top insert is typical of the dyad stage in Kashmir O; bottom insert is typical of the dyad stage in the Canary Islands ecotype). Bottom graph and figures: mean ovule area and depth of the germ-line structure (dyad, ES2 or ES8) for two ecotypes (Kashmir S and Columbia O). Note that the ES2 stage occurs earlier in development (percentage germ-line depth and ovule area) in Kashmire S than in the Columbia O (top insert is typical of the ES2 stage in Kashmir O; bottom insert is typical of the ES2 stage in Columbia O).

FIG. 7. Representative DIC photomicrographs of apomictic embryo sac (aposporous and diplosporous) and autonomous endosperm formation in F₁ hybrids produced by crossing GDS-divergent diploid sexual Antennaria accessions (see FIG. 1 for divergences in GDS timing among Antennaria accessions). A-G, A. umbrinella (MT99003B)×A. racemosa (MT99001D): A, ovule containing a Taraxacum-type diplosporous dyad with vacuolate chalazal member; B, ovule containing an enlarged vacuolate Antennaria-type diplosporous MMC; C, ovule containing megaspores of a degenerating (non-functional) tetrad (identified by the four arrows pointing down and left) that is being consumed by two 1-nucleate vacuolate aposporous embryo sacs of lateral nucellar origin (identified by the other two arrows); D, ovule containing a linear tetrad that is being consumed by a chalazal 2-nucleate aposporous embryo sac of chalazal nucellar origin; E, ovule containing a 3-nucleate aposporous embryo sac of chalazal nucellar origin that is in competition with a 2-nucleate sexual embryo sac; F-G, two focal planes of the same ovule showing early stage autonomous endosperm formation with six endosperm nuclei in the coenocyte. H, A. marginata (NM98015B)×A. racemosa (MT00006D): ovule containing four 1-nucleate aposporous embryo sacs of lateral and terminal origin that are in competition with a 2-nucleate sexual embryo sac. I-J, A. corymbosa (CO00002A)×A. microphylla (CO98030E): ovules containing an enlarged 2-nucleate vacuolate Antennaria-type diplosporous embryo sac.

FIG. 8. Representative DIC photomicrographs of apomictic embryo sac (aposporous and diplosporous) and parthenogenic embryo development in F₁ hybrids produced by crossing GDS-divergent diploid sexual Tripsacum accessions (see FIG. 4 for divergences in GDS timing among Tripsacum accessions). A-B, T. floridanum (TB23.01C)×T. zopilotense (TB42.01B): two focal planes of an ovule containing a degenerating tetrad plus three 1-nucleate aposporous embryo sacs and one 4-nucleate aposporous embryo sac. C, T. floridanum (TB23.01A)×T. dactyloides (TB09.08B): ovule containing a degenerating tetrad (black arrows) and three 1-nucleate aposporous embryo sacs (white arrows). D-F, T. laxum (75-911)×T. pilosum (39-1830) amphiploid (Leblanc et al 1995): D, ovule containing an enlarged 1-nucleate vacuolate Antennaria-type diplosporous embryo sac, E, ovule containing an enlarged vacuolate Antennaria-type diplosporous embryo sac in metaphase of the first mitotic nuclear division, F, ovule containing an 8-12 nucleate (globular stage) parthenogenic embryo (large arrow) and an unfertilized central cell (small arrow).

FIG. 9. Representative DIC photomicrographs of apomictic embryo sac (aposporous and diplosporous) formation in F₁ hybrids produced by crossing GDS-divergent diploid sexual Sorghum accessions (see FIG. 5 for identity of Sorghum accessions and for divergences in GDS timing among accessions). A-B, 5.2×9.2: A, mature sexual MMC; B, enlarged and elongated diplosporous MMC. C, 5.1×4.1: enlarged and elongated diplosporous MMC. D-E, 9.1×1.2: D, degenerating members of a sexual tetrad (black arrows) being replaced by a 2-nucleate aposporous embryo sac (white arrows point to nuclei); E, degenerating members of a sexual tetrad (black arrow) being replaced by a 1-nucleate aposporous embryo sac (white arrows point to nucleus, double white arrows point to vacuoles). F, (5.2×9.2)×5.2: degenerating members of a sexual tetrad (black arrows) being replaced by a 1-nucleate aposporous embryo sac (white arrows). G-H, 2.1×1.1: two focal planes of the degenerating members of a sexual tetrad (black arrows) being replaced by a 2-nucleate aposporous embryo sac (G: white arrows point to nuclei, double white arrows point to vacuoles) in competition with the 1-nucleate sexual embryo sac (H: white arrow points to the nucleus, black arrows point to degenerating megaspores). I, 7.4×9.2: multiple aposporous embryo sacs. (Carman and Naumova, in preparation)

FIG. 10. Process for producing apomictic plants from sexual plants as taught in WO 98/33374. At the beginning of stage 1, genome II produces precocious signals for meiosis, which fail because the archespore mother cell has not yet formed, i.e. it develops at an intermediate rate dictated by the intermediate phenotype. At the beginning of stage 2, end-of-meiosis check-point signals from genome II terminate meiosis and synchronize genome I with genome II in a manner similar to that observed in asynchronous yeast heterokaryons. If meiosis is successfully terminated, one of several forms of diplospory occurs, i.e. an embryo sac forms directly from the megasporocyte (Antennaria-type diplospory) or young female meiocyte (Taraxacum or Ixeris-types of diplospory). If meiosis is unsuccessfully terminated, apospory may occur, i.e. one or more embryo sacs may form from adjacent nucellar cells. This occurs primarily in species containing multiple or ill-defined archegonial cells. In both apospory and diplospory, a genetically unreduced embryo sac develops. Development of the sporophytic tissues of the ovule and ovary continues to occur according to the intermediate-phenotype (delayed) schedule. In contrast, gametophyte (embryo sac) development continues to occur precociously because the embryo sac development genes of genome I (in the embryo sac only) are synchronized with those of genome II. Signals from the two synchronized genomes induce egg formation and parthenogenesis, both of which occur precociously in most apomicts relative to the development of sporophytic tissues of the ovule and ovary. Pollination occurs according to the intermediate phenotype schedule, but the egg is no longer receptive and in many cases has already divided. The central cell, if not autonomous, is fertilized, and the endosperm and parthenogenetic embryo develop (WO 98/33374).

FIG. 11. Mean inner integument lengths at meiosis (M) and the 1-nucleate embryo sac stage for two Sorghum bicolor parent lines (5.1, 7.1), an F₁ hybrid produced there from (1184A.04), and 21 segregating F₂ progeny (see FIG. 5 for parent line origins). Note that transgressive segregation (Rieseberg et al, 2003) has occurred for ES1 onset timing in the 134 F₂, i.e. ES1 onset timing in 134 is earlier than either of its grandparents. Note also that transgressive segregation has occurred for meiosis onset timing in the 150 F₂ and the 12 F₂, i.e. meiosis occurs later in these plants than in either of their grandparents.

FIG. 12. First order inverse regression of the percentage of ovules that express aposporous embryo sac formation vs the difference in integument length between meiosis (dyad and tetrad stages) and the 1-nucleate embryo sac stage for 21 F₂ progeny (see FIG. 11). As the time interval between meiosis and 1-nucleate embryo sac formation decreases, due to genetic factors, the tendency for aposporous embryo sac formation increases. (Carman and Naumova, in preparation)

FIG. 13. Linear regression of percentage abortion of embryo sac development vs the difference in integument length between meiosis (dyad and tetrad stages) and the 1-nucleate embryo sac stage for 21 F₂ progeny (see FIG. 11). Abortion of embryo sac development generally occurs shortly after tetrad formation, i.e. during the period between meiosis and when the 1-nucleate embryo sac would normally form. As the time interval between these two events increases, due to genetic factors, the tendency for early abortion of embryo sac development also increases. (Carman and Naumova, in preparation)

FIG. 14. Representative DIC photomicrographs of aposporous embryo sac formation, tetrad or early embryo sac degeneration, and tetrad polarity reversal in F₂s produced from 1184A.04 (see FIG. 11). A-F, aposporous embryo sac formation: A, ovule containing four aposporous initials (enlarged nucellar cells) and a 1-nucleate sexual embryo sac; B, ovule containing a tetrad in early degeneration and a large 1-nucleate aposporous embryo sac (right of tetrad); C, ovule containing a tetrad in late degeneration and a large 1-nucleate aposporous embryo sac (below and to the right of tetrad); D, ovule containing a tetrad in late degeneration and three 1-nucleate aposporous embryo sacs (right of tetrad); E, ovule containing a tetrad in late degeneration and a large 1-nucleate aposporous embryo sac (right of tetrad); F, ovule containing a tetrad in very late degeneration and a large 2-nucleate aposporous embryo sac (right of tetrad). G-J, tetrad or early sexual embryo sac degeneration: G, degeneration after tetrad formation with some evidence that the functional megaspore had begun to form; H, degeneration at tetrad stage with no evidence of functional megaspore development; I, degeneration apparently after the sexual embryo sac had begun to form, likely at the 1 or 2-nucleate stage; J, degeneration possibly as late as the 4-nucleate sexual embryo sac stage. K, tetrad polarity reversal with the micropylar member having formed a 4-nucleate sexual embryo sac with the other tetrad members forming 1-nucleate sexual embryo sacs. ai=aposporous initial, es1=1-nucleate sexual embryo sac, aes1 or aes2=1 or 2-nucleate aposporous embryo sac. (Carman and Naumova, in preparation)

FIG. 15. Diagrams and DIC photomicrographs of i) sexual megasporogenesis and embryo sac formation in Sorghum, ii) apomictic (aposporous) embryo sac formation in a synthetic apomict produced from sexual Sorghum plants by the methods of the instant specification (F₂ from a cross of two Sorghum bicolor lines, NSL 3999 and PI 542649), and iii) diplosporous embryo sac formation in a synthetic triploid (3×) apomict produced from sexual Tripsacum diploid plants by the methods of the instant specification (T. laxum/T. pilosum 4× amphiploid//T. laxum 2×). Approximate percentages of ovules that produced apomictic embryo sacs were 25% and 90% for the synthetic Sorghum and Tripsacum apomicts, respectively. Note that the somatic number of chromosomes (2n) is reduced to 1n in the sexual process but is maintained at 2n in the nuclei of apomictically-produced embryo sacs. In apospory, the sexual process of producing an embryo sac aborts (crossed out in diagram) and is replaced by a process in which a somatic cell of the nucellus, or rarely the integument, redifferentiates and develops into a genetically-unreduced embryo sac. In diplospory, the megaspore mother cell fails to complete or in some cases even begin meiosis (depending on the type of diplospory, meiotic steps crossed out in diagram), redifferentiates, and develops directly into a genetically-unreduced embryo sac.

FIG. 16. An example from Antennaria of the origins of apomixis according to the reticulate-evolution structural-heterozygosity (RS) model. The diagram depicts divergent GDS phenotypes observed among sexual progenitors of apomictic Antennaria rosea (see FIG. 1-3). GDS phenotypes are encoded by coadapted gene complexes that evolved by habitat speciation processes. Secondary contact hybridization followed by reticulate evolution then shuffled divergent alleles that affect GDS timing. Three segregant classes are observed: 1) those with unresolved GDS asynchronies, which cause sexual sterility or semisterility (outbreeding depression; see FIGS. 11, 14), 2) those with resolved GDS asynchronies, which are sexually fertile (see FIGS. 11, 15), and 3) those with unique ESDS asynchronies, which cause apomixis (asexual fertility, see FIGS. 11, 14). In the latter (apomixis), embryo sac formation preempts meiosis and embryony preempts fertilization. Structural heterozygosity or sexual sterility is then required for apomixis to become genetically stabilized (FIG. 17; WO 01/32001). It is structural heterozygosity that causes apomixis to mimic simple inheritance.

FIG. 17. Karyotypic mechanisms that stabilize the linkage disequilibrium that causes apomixis. For genetic stability to exist, linkage disequilibrium at numerous linked loci must be maintained (see FIG. 11-13), not only through generations of progeny produced asexually (via apomixis) but through generations of progeny facultatively produced through normal sexual processes. The mechanisms diagramed effectively accomplish this stabilization by effectively linking hundreds to thousands of genes together into complex loci (supergene clusters) in which recombination is suppressed.

### DETAILED DESCRIPTION OF THE PREFERRED EMBODIMENTS

### Terminology

Before the present methods of increasing the frequency of apomixis expression in angiosperms are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the instant specification will be limited only by the appended claims and equivalents thereof.

The publications and other reference materials referred to herein are added to describe the background of the invention and to provide additional detail regarding its practice. The references discussed herein are provided solely for their disclosure prior to the filing date of the present application. Nothing herein is to be construed as an admission that the inventor is not entitled to antedate such disclosure by virtue of prior invention.

It must be noted that, as used in this specification and the appended claims, the singular forms “a,” “an,” and “the” include plural referents unless the context clearly dictates otherwise.

In describing and claiming the instant specification, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

As used herein, “apomixis” and grammatical equivalents thereof refer to asexual reproduction of plants through seed. Primarily in older literature, the term apomixis has included reproduction through vegetative structures other than seeds. However, during the past 50 years, the term “apomixis” has increasingly been restricted in the literature to asexual seed formation, including gametophytic apomixis (apospory and diplospory) and forms of adventitious embryony that result in asexual seed formation (Asker and Jerling 1992). In the instant specification, the restricted definition of the term “apomixis” is used. Accordingly, apomictically-produced seeds of apomictic plants contain embryos that are generally genetic clones of the mother plant.

As used herein, “facultative apomict” and grammatical equivalents thereof refer to a solitary plant that reproduces both sexually and apomictically, i.e. one or more ovules of the plant may produce seed sexually and one or more ovules of the plant may produce seed apomictically. With few exceptions all angiospermous apomicts are considered to be facultative apomicts (Asker and Jerling 1992).

As used herein, “obligate apomict” and grammatical equivalents thereof refer to a solitary plant that reproduces only by apomixis. It is believed that few if any obligate plant apomicts exist in nature (Asker and Jerling 1992).

As used herein, “levels of apomixis” and grammatical equivalents thereof refer to the percentage of ovules of a plant that produce seed apomictically. Most of the ovules of a highly or strongly apomictic plant produce seed apomictically. Generally, more than 98% of the ovules of a near-obligate apomictic plant produce seed apomictically. Few ovules of a weakly apomictic plant produce seed apomictically.

As used herein, “genetically-unstable apomictic plant” and grammatical equivalents thereof refer to an apomictic plant in which the level of apomixis among sexually-derived progeny of said genetically-unstable apomictic plant is on average lower than said genetically-unstable apomictic plant. Facultative apomictic reproduction tends to be replaced by sexual reproduction or sterility after several sexually-derived generations starting from a genetically-unstable facultative apomict (WO 01/32001).

As used herein, “MMC” and grammatical equivalents thereof refer to the megaspore mother cell (megasporocyte) in the ovule of an angiospermous plant.

As used herein, “GDS components” and grammatical equivalents thereof refer to components of the germline development sequence. These components consist of MMC differentiation, megasporogenesis, embryo sac formation, fertilization, embryony, and endosperm formation.

As used herein, “elements of apomixis” and grammatical equivalents thereof include unreduced embryo sac formation (aposporous and/or diplosporous), parthenogenesis and/or adventitious embryony, and autonomous or pseudogamous endosperm formation.

### Historical Background

Apomixis occurs naturally in only a few crops and in close relatives of only a few other crops. It occurs in cultivated species or closely-related wild species of sugar cane, citrus, apples, pears, mangos, blackberries, raspberries, walnuts, strawberries, sunflowers, beets, cucumbers and onions. Among forage and turf crops, it occurs in wild or cultivated species of Poa (Kentucky bluegrass), Brachiaria, Bouteloua, Elymus, Cenchrus, Eragrostis, Panicum, Pennisetum, and Paspalum (Carman 1997). The instant specification is directed toward inducing or enhancing the expression of apomixis in such crops as well as all other angiospermous crops that may not have close apomictic relatives. It should be appreciated that the instant specification has additional commercial applications, e.g. in such fields as horticulture, floriculture and forestry (particularly hardwoods).

Apomictic ovule development in angiosperms often begins with megasporogenesis being preempted by a precocious embryo sac formation that initiates from the MMC itself (diplospory) or from a nearby cell of the nucellus (tissue that surrounds the MMC) or rarely the integument (leafy structure that surrounds the nucellus, apospory, FIG. 15). Diplosporous and aposporous embryo sacs contain genetically-unreduced eggs and nuclei (FIG. 7-9, 14). In apomicts, fertilization of the genetically-unreduced egg is generally preempted by precocious embryo formation from the egg (parthenogenesis) or rarely from another unreduced cell of the embryo sac. These events often occur before the stigma is receptive to pollen. Apomictic development may also begin with adventitious embryony wherein a somatic cell of the nucellus or integument develops into an embryo that effectively replaces the sexually-produced egg or embryo. Apomictic development concludes with (1) autonomous (no fertilization of the central cell) or pseudogamous (fertilization of the central cell by one or both sperm nuclei) endosperm formation in a genetically-unreduced aposporous or diplosporous embryo sac, or (2) normal endosperm formation (fertilization of the central cell by a single sperm nucleus) in a genetically-reduced embryo sac wherein the sexual embryo is replaced by an adventitious embryo (Asker and Jerling 1992).

Wobble in the intensity of signals that cause apomixis allows for the facultative expression of sexual reproduction in apomicts. Most if not all apomicts are facultative, which means a certain percentage of seeds produced by the apomict will form sexually, rather than apomictically, and this percentage is influenced by genetic and environmental factors (Asker and Jerling 1992). In a near-obligate apomict, the percentage of seeds per plant that form sexually may be less than 1%. In contrast, weak facultative apomicts may produce less than 1% of their seed apomictically.

Several types of apomixis have been described. Most of these were discovered in the early part of the last century. In Antennaria-type diplospory, signals for precocious embryo sac formation occur very early and cause the MMC, which normally undergoes megasporogenesis, to form a genetically-unreduced embryo sac with no trace of megasporogenesis having been initiated. In Taraxacum-type diplospory, signals for embryo sac formation are less precocious and disrupt megasporogenesis after the first meiotic division. Wobble in the onset time of embryo sac formation in apomictic Elymus rectisetus allows for sexual development, Taraxacum-type diplospory, Antennaria-type diplospory, and various forms that are intermediate between the Taraxacum and Antennaria-types (Crane and Carman 1987). In Hieracium-type apospory, cells affected by precocious and ectopic embryo-sac-inducing signals are located in the nucellus or rarely the integument(s). The affected nucellar or integumentary cell undergoes three rounds of endomitosis to produce a mature genetically-unreduced 8-nucleate embryo sac. In Panicum-type apospory, only two rounds of endomitosis occur resulting in a mature genetically-unreduced 4-nucleate embryo sac. Additional types of apomixis have been described and are reviewed by Asker and Jerling (1992).

Five models of inheritance for apomixis have been hypothesized during the past 100 years:
1. the chromosomal non-homology (wide hybridization) model, in which little or no gene action is required for apomixis;
2. the quantitative inheritance model, in which numerous mutation-derived apomixis genes are required for apomixis;
3. the simple inheritance model, in which one to a very few mutation-derived apomixis genes are required for apomixis;
4. the epigenetic model, in which apomixis genes are not required for apomixis;
5. the reticulation-derived structural-heterozygosity (RS) model, in which unique combinations of wild-type alleles cause apomixis and are stabilized by structural heterozygosity (the inventor's model).

The chromosomal nonhomology model was championed by Ernst in the early part of the 20^{th} century. It stated that apomixis is a function of chromosomal nonhomology, i.e. it is one of several cytogenetic anomalies caused by wide hybridization. Accordingly, apomixis is not controlled by genes, but is a consequence of divergence in chromosome structure. This hypothesis was abandoned shortly after its inception because apomixis was shown to occur in plants whose chromosomes were largely homologous. Later in the century, genetic studies suggested that apomixis is simply inherited, i.e. that it involves genes (Carman 1997).

The quantitative-mode-of-inheritance model was popular in the early to mid 20 h century. It was championed by Muntzing who believed apomixis resulted from a delicate balance of few to many recessive genes, and by Powers, who believed that recessive genes caused three major elements of apomixis: failure of megasporogenesis, apomictic embryo sac formation, and parthenogenesis. In the latter half of the 20^{th} century, results from numerous genetic analyses of apomixis, involving many natural apomicts, strongly implied that apomixis is simply inherited. Hence, the quantitative inheritance hypotheses were largely abandoned (Asker and Jerling 1992).

From the 1960s to the present, most apomixis scientists have favored simple inheritance models, i.e. that one or two dominant genes confer apomixis. Until recently, this conclusion appeared well founded in that Mendelian analyses repeatedly produced simple inheritance segregation ratios, e.g. 1:1 ratios of apomictic to sexual progeny had often been produced in crosses made between sexual and apomictic plants (Asker and Jerling 1992; Savidan 2000, 2001; Sherwood 2001). Based on these findings, several well-funded R&D programs were initiated in the 1980s and 1990s that have focused on transferring the “apomixis gene(s)” from a wild naturally apomictic species to a related sexual crop species through introgressive plant breeding schemes (crossing and backcrossing). However, despite seemingly simple inheritance, years of effort have not resulted in the identification or isolation of the hypothesized “apomixis gene” nor in its transfer to sexual crops to produce commercially-viable crop plants. While apomictic hybrids between apomictic wild relatives and crop species have been readily produced, such hybrids and their backcross progenies have remained weedy or largely sterile (Spielman et al 2003). Furthermore, mapping studies are now indicating that if apomixis is controlled by one or a few apomixis genes, such genes are located in large chromosomal regions in which recombination is suppressed (Spielman et al 2003; Koltunow and Grossniklaus 2003). Consequently, many scientists today are considering the possibility that apomixis may be controlled by numerous genes and modifiers (Carman 1997; WO 98/33374; WO 01/32001; Grimanelli et al 2001; Grossniklaus et al 2001; Richards 2003; Spielman et al 2003; Koltunow and Grossniklaus 2003).

The epigenetic model suggests that apomixis is caused by heritable epigenetic changes in gene regulation. The epigenetic changes are caused by changes in DNA methylation, which may accompany structural changes in chromatin due to hybridization, chromosomal rearrangements and polyploidy. This model combines elements of the mutation and hybridization models. Epialleles are heritable, like mutations, and they can be induced by hybridization and polyploidization (Koltunow and Grossniklaus 2003). However, the epigenetic model does not explain the fact that hybridization and polyploidization have played major roles in the evolution and speciation of over 460 angiospermous families (Ramsey and Schemske 1998), yet over 75% of the genera that contain apomictic species belong to only three families (Carman 1997).

The RS model was developed by the inventor of the instant specification and states that apomixis is the product of multiple quantitatively-inherited traits that are genetically stabilized by structural heterozygosity (FIG. 16-17). Many elements of the RS model were previously verified. For example by hybridizing ecotypes that express divergence in GDS timing, apomictic plants can be produced (WO 98/33374), and by preventing recombination of the causal loci, apomictic plants can be genetically stabilized (WO 01/32001). Further elements of the RS model were discovered after WO 98/33374 and WO 01/32001 had been filed and are elucidated for the first time herein. These elements permit the production of plants that undergo a higher frequency of apomixis expression than plants from which they are produced (FIG. 11-12). In this regard, a patent is being requested herein to protect methods of predictably producing, from sexual or facultatively-apomictic plants, progeny plants that express an increased percentage of apomixis, i.e. plants in which a greater percentage of ovules develop apomictically.

### Enabling Discoveries

The two previous patent applications, WO 98/33374 and WO 01/32001, and the new technology described in the instant specification represent major advances in the state-of-the-art for producing apomictic plants. These patent applications are unique from other published approaches to producing apomictic plants in that the inventor's procedures involve the manipulation of naturally occurring genetic variability for initiation times and durations of megasporogenesis, embryo sac formation, stigma exertion, fertilization, embryony and endosperm formation, all of which are normal sexual processes (FIG. 1-6, 11-13). In contrast, the other published approaches involve the manipulation of a hypothesized apomixis gene(s) of mutagenic origin or the manipulation of epigenetic changes in gene regulation (Spielman et al 2003; Koltunow and Grossniklaus 2003).

The instant specification and WO 98/33374 are similar to each other in that both involve methods of producing plants that express a higher frequency of apomixis than the starting plants. They fundamentally differ with regard to how megasporogenesis, embryo sac formation, fertilization, embryony and endosperm formation are uncoupled so as to permit apomixis to occur. In WO 98/33374, the methods largely rely on asynchrony of entire GDS sequences (FIG. 10). The instant specification relies on procedures that temporally uncouple (a) megasporogenesis from embryo sac formation so that aposporous or diplosporous embryo sac formation may occur (embryo sac formation encoded at the same time as or before megasporogenesis) and (b) egg maturation, central cell formation and maturation, and fertilization from embryony and endosperm formation so that parthenogenesis and autonomous or pseudogamous endosperm formation may occur (FIG. 11-16).

Before elucidating the specific processes of the instant specification, it is desirable to review current hypotheses concerning genes that regulate the various elements of apomixis. By comparing these hypotheses with the inventor's discoveries, it is clear that the inventor's processes materially advance the state-of-the-art with regard to the development of methods for predictably producing apomictic plants.

The elements of apomixis were clearly described in the first half of the 20^{th} century and include disruption or abortion of megasporogenesis, aposporous or diplosporous embryo sac formation, parthenogenesis or adventitious embryony, and autonomous or pseudogamous endosperm formation (Gustafsson 1946, 1947a, 1947b). Clearly, the most popular school of thought during the past 40 years, concerning the genetic regulation of apomixis, is that a single mutation that causes aposporous or diplosporous embryo sac formation could pleiotropically cause parthenogenesis as well (Savidan 2000, 2001). However, many scientists, starting with several in the first half of the 20^{th} century, have genetically uncoupled apomictic embryo sac formation from parthenogenesis, which suggests that these elements of apomixis are regulated by separate genes (Gustafsson 1947a; Spielman et al 2003). This understanding of an uncoupling of discrete developmental steps, which was first arrived at in the first half of the 20^{th} century (Gustafsson 1947a), was later memorialized by Nogler, who extended the idea by describing apomictic embryo sac formation as an opening (or uncoupling) of the developmental bonds linking megasporogenesis with sexual embryo sac formation (Nogler 1984).

Most scientists who study apomixis today believe that the uncoupling and reshuffling events that cause apomixis are controlled in most cases by at least two apomixis genes (mutations of normal genes), which are generally tightly linked, or by epigenetic modifications to the regulation of wild-type genes (Spielman et al 2003; Koltunow and Grossniklaus 2003). In contrast, the inventor of the instant specification has demonstrated that apomixis is the result of competition between asynchronously-expressed developmental programs that are combined together either naturally or intentionally by hybridization. As taught in WO 98/33374, apomictic embryo sac formation may occur when genes that initiate sexual embryo sac formation (inherited from a plant wherein megasporogenesis, embryo sac formation, fertilization, embryony and endosperm formation occur early in ovule development) are expressed at about the same time as or earlier than genes that initiate megasporogenesis (inherited from a different plant wherein megasporogenesis, etc, occur relatively late in ovule development) (FIG. 10). Hence, apomixis is not caused by apomixis genes per se, or by epigenetic modifications, but is an inherited trait wherein wild-type alleles responsible for an early embryo sac formation compete with and preempt wild-type alleles for a late megasporogenesis, and wild-type alleles for an early embryony and endosperm formation compete with and preempt wild-type alleles that encode a requirement for egg and central cell maturation and fertilization relative to the developmental maturity of sporophytic ovule tissues.

It was taught in WO 01/32001 that sexually-derived segregants of a facultatively apomictic plant would express either the same level of apomixis as the parent plant (same percentage of ovules developing apomictically) or a lower level. For example, recombination involving heterozygous loci responsible for apomixis (asynchronously expressed female developmental sequences) could, if inherited, result in progeny wherein the critical loci are homozygous, which could cause a disruption of the developmental competition responsible for apomixis. In this respect, the uncoupling of megasporogenesis and fertilization were viewed only as a preemptive removal of these processes by competing signals (FIG. 10). In WO 98/33374, it had not yet been contemplated or proposed that initiation times and durations of megasporogenesis, embryo sac formation, fertilization, etc, are regulated independently of each other. In contrast, it was taught that asynchrony involved the entire sequence (FIG. 10) not individual components of the sequence (FIG. 11-14, 16).

The inventor recently discovered that genes controlling initiation times and durations of megasporogenesis are different from genes controlling initiation times and durations of embryo sac formation, etc (FIG. 11-14). Based on this discovery, the inventor theorized that (1) MMC formation, megasporogenesis, embryo sac formation, fertilization, embryony and endosperm formation can be genetically uncoupled from each other through breeding and selection for initiation times and durations of each step individually, and (2) sexually-recombinant progeny from a genetically-unstable apomict may express higher levels of apomixis if genetic segregation reshuffles alleles such that early megasporogenesis alleles are lost while alleles for early embryo sac formation are retained (FIG. 11-13). In testing this theory, it was verified that a low frequency of sexual segregants from synthetically-produced and genetically-unstable facultatively-apomictic plants express much higher frequencies of apomictic ovule development than the mother plant (FIG. 11-12). This phenomenon is predictable and is a novel discovery of the inventor.

The instant specification, in combination with WO 98/33374 and WO 01/32001, provide methods of producing genetically-stabilized apomictic plants from sexual or facultatively-apomictic plants, the latter being less apomictic than the apomictic plants produced. High frequency apomixis (near obligate expression) is important for many agricultural applications of apomixis. Near obligate expression assures crop uniformity (flowering date, plant height, yield variables, nutrition variables, etc), which is necessary for modern agricultural practices. Consequently, it is advantageous to provide methods that result in plants with an increased frequency of apomixis expression relative to the plants from which the improved lines were derived. It should be appreciated that these and other advantages of the present application (discussed below) represent major advancements in the state-of-the-art.

### Processes of the Instant Specification

It is convenient to separate the processes of the instant specification into five categories: (a) compiling groups of lines that contain sufficient genetic variability to produce, through plant breeding or genetic engineering, plants in which the within-plant frequency of expression of apomixis (or one or more of its elements) is increased, (b) selfing, crossing or otherwise genetically-modifying said groups of lines in such a way as to produce plants from which more highly apomictic plants may be obtained, (c) producing subsequent-generation recombinant plants, (d) screening plants for apomixis, and (e) repeating certain steps to increase the frequency with which apomictically-enhanced plants are produced.

Compiling Groups of Lines from which Plants with Enhanced Levels of Apomixis May be Produced

One step of the methods involves selecting or producing sexual or facultatively-apomictic plants from which plants with an increased expression of apomixis can be produced. One process for obtaining said sexual or facultatively-apomictic plants is to follow the methods of WO 98/33374. Accordingly, the parent plants of each said sexual or facultatively-apomictic plant will have been delineated such that initiation of embryo sac formation in one parent occurs at about the same time as or before megasporogenesis in the other parent relative to developmental maturity of sporophytic ovule or ovary tissues (FIG. 1-6, 10).

Another step involves choosing said related plants so that they contribute genetic variability (different alleles) to the next generation of hybrids with regard to initiation times and durations of megasporogenesis, embryo sac formation, fertilization, embryo formation, or endosperm formation relative to sporophytic ovule or ovary tissues (FIG. 1-6, 11-13, 16). For example, if embryo sac formation in one parent of said sexual or facultatively-apomictic hybrid plant occurs at about the same time as or before megasporogenesis in the other parent relative to developmental maturity of sporophytic ovule and ovary tissues, but fertilization in both parent plants occurs at about the same time relative to developmental maturity of sporophytic ovule and ovary tissues, then plants in which fertilization occurs much earlier or much later than the two parents could be chosen as said related plants.

It should be appreciated that genetic variability for initiation times and durations of the various stages of female development in ovules is a new discovery of the inventor (FIG. 1-6, 11-13), and that apomixis appears to have evolved in nature as a consequence of reticulate evolution (FIG. 16), involving the segregational recombination of genes controlling initiation times and durations of ovule development stages, followed by genetic stabilization as described in WO 01/32001 (see FIG. 17). Hence, as stated above, an object of the instant specification is to provide methods for enhancing genetic variability within individual plants or groups of plants for initiation times and durations of megasporogenesis, embryo sac formation, fertilization, embryo formation and endosperm formation from which plants with enhanced expressions of apomixis may be produced.

Genetically-Modifying Groups of Lines to Increase the Frequency in which Plants with Enhanced Levels of Apomixis are Produced

Another step of the methods involves either crossing or selfing said sexual or facultatively-apomictic hybrid plants or outcrossing one or more of them to a related plant of the same species, genus or family. It is anticipated that genes controlling timing of GDS stages will soon be cloned, and these could be used in an alternative approach involving transformation to modify GDS timing in appropriate ways so as to induce apomixis. For plant breeding, standard plant breeding procedures may be used to accomplish selfing, crossing or outcrossing such as are taught in Poehlman (1987), and for mapping, cloning and transformation, standard approaches well practiced in the industry may be used such as are taught in Weigel and Glazebrook (2002).

Producing Subsequent-Generation Recombinant Plants from which Plants with Enhanced Levels of Apomixis May be Selected

Other steps of the methods include sowing seed obtained from selfing, backcrossing, cross-hybridizing (e.g., full-sib or half-sib crossings, see Poehlman 1987), outcrossing or genetic engineering and growing the resulting second-generation or later generation plants.

Selecting Plants with Enhanced Levels of Apomixis

Additional steps of the methods include screening first, second or subsequent generations of plants for apomixis by (a) using cyto-embryological procedures (FIG. 7-9, 11-14) or (b) producing progeny of the first, second or subsequent generations of plants and progeny testing the plants to identify apomictic plants that express a higher frequency apomixis than their parents.

Repeating Certain Steps to Increase the Frequency with which Apomictically-Enhanced Plants are Produced

Further steps of the methods include repeating one or more times prior steps to produce additional segregating generations from which plants with enhanced apomixis are derived and identified by conducting additional screening and progeny test procedures.

### EXAMPLES

The present invention is described by reference to the following examples. These are offered by way of illustration only. They are not intended to limit the invention in any manner. Standard techniques well known in the art or the techniques specifically described below were used. It will be appreciated that the instant specification may be embodied in many specific forms without departing from its spirit or essential characteristics.

### Example 1

### Selecting Antennaria Lines from which Apomixis-Enhanced Plants May be Produced

Apomixis was first described at the embryological level in Antennaria alpina (Juel 1900). Antennaria (x=14) are dioecious, herbaceous perennials and are usually stoloniferous. Morphology-based cladistic analyses of 32 sexual diploid species coupled with analyses of sequenced internal transcribed spacer regions of nuclear ribosomal DNA (ITS-1 & ITS-2) indicated that Antennaria is composed of six clades (Bayer 1990; Bayer et al 1996). Apomixis occurs only in the Catepes clade, which contains 17 of the 32 sexual Antennaria species and sexual and apomictic polyploids ranging from 4× to 12× (Bayer and Stebbins 1987; Bayer and Minish 1993). All members of this group are stoloniferous and sexually dimorphic. Five geographically-divergent complexes of interbreeding sexual and apomictic Antennaria species (agamic complexes), A. alpina (L.) Gaertn., A. howellii E. L. Greene, A. parlinii Fern., A. parvifolia Nutt., and A. rosea, have evolved from among the sexual members of the Catipes (Bayer 1987; Bayer et al 1996).

TABLE 1
Antennaria sp, number of sites collected and latitudinal range.

| Species | Sites | Latitudinal range |
|---|---|---|
| A. alpina | 1 | 64° 32′ |
| A. aromatica | 6 | 44° 46′-47° 54′ |
| A. corymbosa | 6 | 38° 01′-46° 14′ |
| A. densifolia | 3 | 64° 57′-65° 24′ |
| A. friesiana alaskana | 3 | 63° 48′-66° 58′ |
| A. friesiana friesiana | 2 | 63° 14′-64° 05′ |
| A. friesiana neoalaskana | 1 | 67° 05′ |
| A. marginata | 10 | 32° 56′-36° 39′ |
| A. media | 3 | 37° 11′-50° −36′ |
| A. microphylla | 11 | 37° 10′-53° 05′ |
| A. monocephala | 7 | 61° 46′-65° 29′ |
| A. parvifolia | 7 | 35° 17′-38° 01′ |
| A. racemosa | 12 | 43° 19′-51° 26′ |
| A. rosea | 21 | 36° 33′-64° 28′ |
| A. rosulata | 8 | 35° 17′-38° 12′ |
| A. umbrinella | 13 | 40° 56′-49° 10′ |

The center of diversity for the A. rosea agamic complex is the Rocky Mountains of North America, and its range is from New Mexico and southern California, north to Alaska and the Northwest Territories, and east through Alberta, Saskatchewan, the northern Great Lakes and with disjunct populations in Atlantic Canada (Bayer 1989a). It is generally tetraploid, although triploid and pentaploid plants have been found (Bayer and Stebbins 1987). It appears to have arisen by multiple hybridizations and introgression involving A. aromatica Evert, A. corymbosa E. Nelson, A. pulchella E. Greene, A. marginata E. Greene, A. microphylla Rydb., A. racemosa Hook., A. rosulata Rydb. and A. umbrinella Rydb. These are sexual species that primarily inhabit unglaciated portions of the western cordillera. Phenetic analyses indicated that A. aromatica, A. corymbosa, A. microphylla, A. pulchella/media, and A. umbrinella are the major sexual progenitors of the A. rosea complex. Only a few A. rosea clones displayed morphological characteristics that can be attributed to A. marginata or A. rosulata (Bayer 1990). Analyses of all pairwise comparisons of zymograms from 33 A. rosea populations indicated only slight divergence. The genetic identity (I) averaged 0.944 (range=0.802-0.997). Most of the A. rosea populations were most similar to populations of A. corymbosa, A. microphylla, A. pulchella/media and A. umbrinella. Fewer were similar to A. aromatica, A. marginata, and A. rosulata (Bayer 1989b).

In an extensive survey of biotic and abiotic factors, the sexual A. aromatica, A. corymbosa, A. marginata, A. media/pulchella, A. microphylla, A. racemosa, A. rosulata, and A. umbrinella occurred in distinct habitats but apomictic A. rosea occupied the center of the overall ordination and overlapped at least parts of the ordination space of all sexual species except A. aromatica and A. racemosa (Bayer et al 1991). This study further supported the hypothesis of a multiple hybrid origin for A. rosea. Many sites of A. rosea occupy similar habitats to their diploid sexual progenitors, and other sites occupy hybrid habitats that are intermediate between those of their sexual progenitors (Bayer et al 1991). Hence, there is good evidence from a variety of sources (morphologic, isozymic, ecological) to support the hypothesis of a multiple hybrid origin for the A. rosea agamic complex.

Sexual Antennaria were collected from their native habitats throughout the Rocky Mountain Cordillera (Table 1), grown in cultivation, embryologically analyzed for GDS variation (FIG. 1-3) and bred to produce plants that express apomixis (FIG. 7).

### Example 2

### Selecting Sorghum Lines from which Apomixis-Enhanced Plants May be Produced

There is evidence that low level facultative apomictic seed formation (up to 25%) has occurred in at least some Sorghum lines (Hanna et al 1970; Tang et al 1980; Schertz 1992; Bala Ravi 1993). To assess whether apomixis in these lines arose from hybridization, rather than fortuitous mutation, we tested the following null hypothesis: apomixis fails to arise in hybrids produced by crossing progenitors of known facultatively-apomictic sorghum lines. To our knowledge, such simple tests had not previously been conducted, i.e. conventional wisdom assumed that apomixis arose by mutation. Progenitors of two facultatively-apomictic Sorghum lines, ‘R473’ and ‘302’, were obtained. Progenitors of R473 are ‘IS 2942’ (a day neutral Kafir line) and ‘Aispuri’ (a short day Indian variety) (Tang et al 1980). Progenitors of 302 are ‘IS 3922’ and ‘Karad Local’ (Rana et al 1981). Additional lines totaling 20 S. bicolor, 14 tetraploid Sorghum×Almum, four tetraploid S. halapense, three S. arundinacium, three tetraploid S. australiense (2n=4x=20, i.e. n=5), and two wide hybrids of unknown parentage were chosen based on diversity of habitat or prior history of being involved in apomixis research. GDS-characterized lines involved in producing hybrids that were subsequently screened embryologically for apomixis are listed in Table 2.

TABLE 2
GDS-characterized lines used to produce hybrids that have been screened for apomixis.

| ID# | US # | ICRISAT # | Species | Race | name | origin | Sensitivity | Status |
|---|---|---|---|---|---|---|---|---|
| SB 01001.1 | PI 253638 | IS 1151 | S. bicolor | durra | aispuri | India | S | Landrace |
| SB 01001.2 | PI 533817 | IS 1151C | S. bicolor | | aispuri (C) | India | I | Breeding material |
| SB 01002.1 | NSL 51477 | IS 2942 | S. bicolor | kafir | kafir | | I | Breeding material |
| SB 01003.1 | NSL 51483 | IS 3922 | S. bicolor | kafir-durra | IS 3922 | | I | Breeding material |
| SB 01004.1 | PI 533932 | IS 1122(A)C | S. bicolor | | karad local (C) | India | I | Breeding material |
| SB 01004.2 | PI 248318 | IS 1122 | S. bicolor | durra | karad local | India | S | Landrace |
| SB 01005.1 | NSL 3999 | IS 851 | S. bicolor | durra-caudatum | early kalo | | I | Breeding material |
| SB 01005.2 | NSL 4003 | IS 836 | S. bicolor | durra-caudatum | westland | | I | Breeding material |
| SB 01006.1 | PI 571105 | IS 9683 | S. bicolor | kafir-durra | colby | Sudan | S | Landrace |
| SB 01007.1 | PI 542649 | | S. hybrid | | 1111 | Algeria | I | Landrace |
| SB 01007.3 | PI 302166 | | S. halepense | | 0 756 | Australia | I | Wild |
| SB 01008.1 | PI 225905 | IS 12693 | S. hybrid | | is 12693 | Zambia, Zimbabwe | S | Landrace |
| SB 01008.2 | PI 329251 | | S. arundinaceum | | r-319 | Ethiopia | S | Landrace |
| SB 01009.1 | PI 562347 | | S. bicolor | durra | vir-5049 | Sudan | I | Landrace |
| SB 01009.2 | PI 217855 | | S. bicolor | caudatum | agira | Sudan | S | Landrace |
| SB 01010.1 | PI 229862 | IS 2386 | S. bicolor | kafir | lydenburg red | S. Africa | I | Landrace |

### Example 3

### Characterizing GDS Variation in Sorghum Lines and Producing Plants that Express Apomixis

Pistils for cytological analysis were killed, fixed, cleared and observed using DIC microscopy as in Peel et al (1997a,b). Cytological data was obtained at the MMC, dyad, triad/tetrad, functional megaspore, 1-nucleate embryo sac, 2-nucleate embryo sac, 4-nucleate embryo sac, early 8-nucleate embryo sac, mature embryo sac, stigma exertion, and ripe seed stages. The following data was obtained for each ovule analyzed: meiotic or embryo sac development stage, pistil length and width, integument length and width, and meiocyte or embryo sac length and width. Tables 3-4 exemplify data sheets used to GDS-characterize Sorghum lines from the MMC to mature embryo sac stages (data from line SB1001.1 are shown). Additional data sheets were used to obtain cytological data for the stigma exertion and ripe seed stages. Plants from Table 2 were grown, embryologically analyzed for GDS variation (FIG. 5), and used to produce plants that express apomixis by way of the methods of WO 98/33374 (FIG. 9) and by way of the methods of the instant specification (FIG. 11-15).

Genetic segregation was extensive among the 21 F₂s of two Sorghum bicolor parent lines (5.1, 7.1) for initiation times and durations of meiosis and embryo sac formation relative to the maturity level (length) of the inner integument (FIG. 11). Several F₂s segregated toward one or the other parent, e.g. the inner integument lengths at which M and ES-1 were reached in 245 and 150 were similar to those of the maternal parent, while integument lengths at these stages in 108 resembled the paternal parent. These data provide additional evidence that genes influencing initiation times for meiosis may differ from genes influencing initiation times for embryo sac formation.

The F₂ progeny in FIG. 11 are sorted, in decreasing order, by duration from M to ES-1 (length of the yellow bar). It is an object of the instant specification to emphasize that a decreasing M to ES-1 duration (length of yellow bar) is negatively correlated (highly significant) with percentage aposporous embryo sac formation (top numbers in FIG. 11; see FIG. 12) and positively correlated (significant) with ovule degeneration (bottom numbers in FIG. 11; see FIG. 13). Frequency ovule degeneration and frequency aposporous embryo sac formation were low in the parents. Only 4% of 135 appropriately-staged ovules in 5.1 had formed aposporous initials; none had formed aposporous embryo sacs. In contrast, many of the 9% of ovules in the F₁ hybrid (1184A.04), from which all F₂s were derived, exhibited aposporous embryo sac formation (percentages were based on presence or absence of aposporous activity in ovules that were in the functional megaspore stage to the 2-nucleate embryo sac stage, i.e. stages in which aposporous embryo sac formation is readily observed). In this respect, the majority of F₂s segregated away from aposporous embryo sac formation, relative to their F₁ parent. However, some F₂s retained a similar level of aposporous embryo sac activity (FIG. 11, plants 70, 218, 12), while two F₂s exceeded the F₁ level by 2 to 3-fold (FIG. 11, plants 182 & 134; FIGS. 14, 15). Ovules from various F₂s exhibited anomalies other than aposporous and diplosporous embryo sac formation including tetrad degeneration, polarity reversals of linear tetrads, and multiple members of tetrads undergoing embryo sac formation (FIG. 14). These anomalies may be caused by disharmony among alleles responsible for GDS timing (Carman and Naumova, in preparation).

TABLE 3
Data sheet containing Sorghum bicolor SB1001.1
raw data (summarized in Table 3).

| Count of stage | |
|---|---|
| stage | Total |
| dy | 10 |
| es1 | 11 |
| es2 | 7 |
| es4 | 7 |
| es8 | 14 |
| fms | 18 |
| mes | 10 |
| mmc | 10 |
| tr-tet | 13 |
| (blank) | |
| Grand Total | 100 |

Fixation Inventory Notes
Sorghum bicolor Tag # PI253638 Plant # 1-1 variety Aispuri (tall)
Ovary = dissecting microscope 12X 250 um = 3.25 lines
Ovule and integuments = Olympus microscope 20X 1 um = 0.835 cm

| | | | out | in | ReproSt | | | |
|---|---|---|---|---|---|---|---|---|
| Verify | fix. # | ovary | int | int | length | stage | notes | date |
| | 3 | 4.00 | | 4.00 | 3.00 | dy | mmc- | 16 Jul. 2001 |
| | | | | | | | late proph | |
| | 3 | 3.00 | | 5.00 | 3.00 | dy | mmc (proph) | 16 Jul. 2001 |
| | 3 | 4.00 | | 5.00 | 3.00 | dy | | 16 Jul. 2001 |
| | 3-1c | 4.00 | 1.00 | 6.00 | 1.50 | dy | | 13 Jun. 2000 |
| | 114-1 | 3.00 | 2.00 | 6.00 | 3.00 | dy | | 8 Aug. 2000 |
| | 114-1 | 3.00 | 1.50 | 6.00 | 3.20 | dy | | 8 Aug. 2000 |
| | 108-3 | 3.00 | 2.00 | 6.00 | 3.80 | dy | | 7 Aug. 2000 |
| | 3 | 4.00 | | 6.00 | 3.50 | dy | | 16 Jul. 2001 |
| | 108-2 | 3.50 | 1.00 | 6.50 | 5.00 | dy | fms after | 7 Aug. 2000 |
| | | | | | | | dy | |
| | 108-2 | 4.00 | 2.00 | 7.00 | 4.50 | dy | | 7 Aug. 2000 |
| | 43-1, 2 | 5.00 | 2.50 | 6.50 | 2.50 | es1 | | 23 Jun. 2000 |
| | 3 | 5.00 | | 9.00 | 2.50 | es1 | | 16 Jul. 2001 |
| | 3 | 5.00 | | 11.00 | 2.20 | es1 | | 16 Jul. 2001 |
| | 17-2 | 5.00 | 3.50 | 12.00 | 2.00 | es1 | | 14 Jun. 2000 |
| | 3-1c | 7.00 | 3.00 | 14.00 | 1.50 | es1 | | 13 Jun. 2000 |
| | 43-1, 2 | 5.00 | 4.00 | 14.00 | 2.50 | es1 | | 23 Jun. 2000 |
| | 43-2 | 4.00 | 6.50 | 14.00 | 3.00 | es1 | | 23 Jun. 2000 |
| | 3-1. | 5.00 | 4.00 | 15.50 | 2.30 | es1 | | 2 Aug. 2000 |
| | 3-1. | 5.50 | 5.00 | 15.50 | 3.00 | es1 | | 2 Aug. 2000 |
| | 43-3 | 4.00 | 7.50 | 16.00 | 2.00 | es1 | | 23 Jun. 2000 |
| | 3~3 | 6.00 | 8.50 | 19.50 | 2.00 | es1 | | 14 Jun. 2000 |
| | 43-2 | 5.00 | 4.00 | 14.00 | 3.50 | es2 | | 23 Jun. 2000 |
| | 3-1. | 5.00 | 6.00 | 16.00 | 4.00 | es2 | | 2 Aug. 2000 |
| | 17-2 | 5.00 | 6.00 | 16.50 | 2.30 | es2 | | 14 Jun. 2000 |
| | 3-1. | 5.50 | 7.50 | 16.50 | 4.50 | es2 | | 2 Aug. 2000 |
| | 43-2 | 5.00 | 8.50 | 17.00 | 3.00 | es2 | | 23 Jun. 2000 |
| | 3-1. | 5.50 | 5.50 | 17.50 | 4.20 | es2 | | 2 Aug. 2000 |
| | 43-3 | 6.00 | 9.50 | 18.00 | 5.00 | es2 | | 23 Jun. 2000 |
| | 43-2 | 4.00 | 8.00 | 15.00 | 4.50 | es4 | | 23 Jun. 2000 |
| | 43-1, 2 | 6.00 | 6.00 | 15.00 | 6.00 | es4 | | 23 Jun. 2000 |
| | 3~3 | 5.00 | 5.50 | 15.50 | 4.00 | es4 | | 14 Jun. 2000 |
| | 17-2 | 5.00 | 5.00 | 19.00 | 4.00 | es4 | | 14 Jun. 2000 |
| | 3~2 | 5.00 | 9.00 | 20.00 | 3.20 | es4 | | 13 Jun. 2000 |
| | 5~2 | 5.00 | 8.00 | 20.00 | 5.00 | es4 | | 17 Jul. 2000 |
| | 17-2 | 5.00 | 6.00 | 20.50 | 2.80 | es4 | | 14 Jun. 2000 |
| | 4~2 | 5.00 | 7.50 | 19.00 | 6.50 | es8 | | 17 Jul. 2000 |
| | 43-3 | 7.00 | 9.00 | 20.00 | 6.00 | es8 | | 23 Jun. 2000 |
| | 3-1. | 6.00 | 9.00 | 20.50 | 7.50 | es8 | | 2 Aug. 2000 |
| | 3-1. | 6.00 | 10.00 | 20.50 | 7.50 | es8 | | 2 Aug. 2000 |
| | 43-3 | 6.00 | 11.00 | 20.50 | 8.50 | es8 | | 23 Jun. 2000 |
| | 43-2 | 7.00 | 11.50 | 21.00 | 3.00 | es8 | | 23 Jun. 2000 |
| | 43-3 | 7.00 | 13.00 | 21.00 | 6.00 | es8 | | 23 Jun. 2000 |
| | 17-2 | 5.00 | 6.00 | 21.00 | 6.30 | es8 | | 14 Jun. 2000 |
| | 4~2 | 5.00 | 11.00 | 21.00 | 6.70 | es8 | | 17 Jul. 2000 |
| | 43-3 | 6.00 | 11.00 | 22.00 | 7.00 | es8 | | 23 Jun. 2000 |
| | 3-1. | 6.00 | 9.00 | 22.00 | 7.50 | es8 | | 2 Aug. 2000 |
| | 43-3 | 6.00 | 14.50 | 22.00 | 9.00 | es8 | | 23 Jun. 2000 |
| | 43-3 | 7.00 | 15.00 | 22.50 | 7.00 | es8 | | 23 Jun. 2000 |
| | 43-3 | 7.00 | 12.00 | 23.00 | 8.00 | es8 | | 23 Jun. 2000 |
| | 43-1, 2 | 4.00 | 0.50 | 5.00 | 1.30 | fms | | 23 Jun. 2000 |
| | 114-1 | 3.00 | 3.00 | 5.50 | 1.60 | fms | or triad | 8 Aug. 2000 |
| | 108-3 | 3.50 | 1.50 | 6.00 | 1.30 | fms | | 7 Aug. 2000 |
| | 114-1 | 3.50 | 1.50 | 7.00 | 1.50 | fms | | 8 Aug. 2000 |
| | 108-3 | 4.00 | 2.00 | 8.00 | 1.50 | fms | | 7 Aug. 2000 |
| | 108-3 | 4.00 | 3.00 | 9.00 | 1.50 | fms | | 7 Aug. 2000 |
| | 108-3 | 4.50 | 3.00 | 9.00 | 1.50 | fms | | 7 Aug. 2000 |
| | 114-1 | 4.00 | 3.50 | 9.00 | 1.60 | fms | | 8 Aug. 2000 |
| | 3-1. | 3.50 | 2.00 | 9.50 | 2.00 | fms | | 2 Aug. 2000 |
| | 108-3 | 5.00 | 4.00 | 10.00 | 1.50 | fms | | 7 Aug. 2000 |
| | 108-3 | 5.00 | 3.50 | 10.00 | 1.50 | fms | | 7 Aug. 2000 |
| | 3-1. | 4.50 | 3.00 | 10.50 | 1.50 | fms | | 2 Aug. 2000 |
| | 3-1. | 4.00 | 2.00 | 10.50 | 2.00 | fms | | 2 Aug. 2000 |
| | 3-1. | 4.00 | 2.50 | 11.50 | 1.80 | fms | | 2 Aug. 2000 |
| | 3-1c | 5.00 | 3.00 | 13.50 | 0.80 | fms | | 13 Jun. 2000 |
| | 3-2. | 4.00 | 5.00 | 14.00 | 2.50 | fms | | 13 Jun. 2000 |
| | 3-1. | 5.00 | 5.00 | 15.00 | 2.00 | fms | | 2 Aug. 2000 |
| | 3-2. | 6.00 | 6.00 | 19.00 | 1.80 | fms | | 13 Jun. 2000 |
| | 3-1c | 7.00 | 3.00 | 14.00 | 1.50 | mes | | 13 Jun. 2000 |
| | 3-2. | 5.00 | 7.00 | 18.00 | 5.00 | mes | | 13 Jun. 2000 |
| | 17-2 | 6.00 | 5.00 | 19.00 | 4.00 | mes | | 14 Jun. 2000 |
| | 3-2. | 6.00 | 14.00 | 23.00 | 6.00 | mes | | 13 Jun. 2000 |
| | 3-2. | 6.00 | 7.00 | 23.00 | 7.00 | mes | | 13 Jun. 2000 |
| | 3-2. | 5.00 | 9.50 | 24.00 | 5.00 | mes | | 13 Jun. 2000 |
| | 3-2. | 7.00 | 11.00 | 24.00 | 7.80 | mes | | 13 Jun. 2000 |
| | 43-3 | 7.00 | 16.00 | 25.00 | 10.00 | mes | | 23 Jun. 2000 |
| | 43-3 | 7.00 | 16.00 | 25.00 | 10.00 | mes | | 23 Jun. 2000 |
| | 3-2. | 7.00 | 14.00 | 27.50 | 7.00 | mes | | 14 Jun. 2000 |
| | 108-1 | 3.00 | 0.00 | 2.00 | 1.70 | mmc | | 7 Aug. 2000 |
| Y | 3-1a | 2.50 | 1.00 | 2.50 | 1.00 | mmc | | 13 Jun. 2000 |
| | 108-1 | 2.50 | 0.00 | 2.50 | 1.20 | mmc | | 7 Aug. 2000 |
| y | 3-1a | 4.00 | 10.00 | 4.00 | 1.50 | mmc | | 13 Jun. 2000 |
| | 114-1 | 2.50 | 1.50 | 4.50 | 3.00 | mmc | | 8 Aug. 2000 |
| | 114-1 | 3.00 | 1.50 | 5.00 | 2.50 | mmc | | 8 Aug. 2000 |
| | 114-1 | 2.50 | 1.50 | 5.00 | 3.00 | mmc | | 8 Aug. 2000 |
| | 43-1, 2 | 3.00 | 1.80 | 5.20 | 2.00 | mmc | | 23 Jun. 2000 |
| | 114-1 | 2.50 | 3.00 | 7.00 | 3.50 | mmc | | 8 Aug. 2000 |
| | 3-1. | 4.00 | 2.00 | 8.50 | 2.50 | mmc | | 2 Aug. 2000 |
| | 108-2 | 3.50 | 1.00 | 4.50 | 4.00 | tr-tet | | 7 Aug. 2000 |
| | 108 | 4.00 | | 5.50 | 3.60 | tr-tet | triad | 16 Jul. 2001 |
| | 114-1 | 2.50 | 1.50 | 5.50 | 3.30 | tr-tet | | 8 Aug. 2000 |
| | 108-3 | 3.00 | 2.50 | 6.00 | 1.30 | tr-tet | | 7 Aug. 2000 |
| | 114-1 | 2.50 | 2.00 | 6.00 | 3.50 | tr-tet | | 8 Aug. 2000 |
| | 114-1 | 3.00 | 1.50 | 6.00 | 3.50 | tr-tet | | 8 Aug. 2000 |
| | 108-3 | 3.50 | 2.00 | 6.50 | 4.00 | tr-tet | | 7 Aug. 2000 |
| | 3-1. | 4.00 | 2.20 | 7.20 | 3.50 | tr-tet | tr-tet (late) | 2 Aug. 2000 |
| | 3 | 5.00 | | 7.50 | 3.50 | tr-tet | | 16 Jul. 2001 |
| | 108-2 | 4.00 | 2.00 | 7.50 | 3.70 | tr-tet | | 7 Aug. 2000 |
| | 108-2 | 4.50 | 2.00 | 8.00 | 4.00 | tr-tet | | 7 Aug. 2000 |
| | 3 | 4.00 | | 9.00 | 4.20 | tr-tet | triad | 16 Jul. 2001 |
| | 108-3 | 5.00 | 3.00 | 9.00 | 4.50 | tr-tet | tr-tet (late) | 7 Aug. 2000 |

TABLE 4
Mean lengths and standard deviations of ovaries, outer and inner integuments,
and reproductive structures (meiocyte or ES) of S. bicolor lines for
nine stages of ovule development (3450 ovules total).

| SB01001.1 Average Lengths | | | | | | SB01001.1 Standard Deviations | | | | | |
|---|---|---|---|---|---|---|---|---|---|---|---|
| | | out | | | no. | | | out | | | no. |
| stage | ovary | int | in int | ReproSt | samples | stage | ovary | int | in int | ReproSt | samples |
| mmc | 2.95 | 2.23 | 4.62 | 2.19 | 10 | mmc | 0.60 | 2.87 | 2.04 | 0.84 | 10 |
| dy | 3.55 | 0.95 | 5.75 | 3.35 | 10 | dy | 0.50 | 0.90 | 0.86 | 0.95 | 10 |
| tr-tet | 3.73 | 1.52 | 6.78 | 3.58 | 13 | tr-tet | 0.83 | 0.99 | 1.38 | 0.77 | 13 |
| fms | 4.25 | 3.00 | 10.11 | 1.62 | 18 | fms | 0.73 | 1.38 | 3.55 | 0.36 | 18 |
| es1 | 5.14 | 4.05 | 13.36 | 2.32 | 11 | es1 | 0.84 | 2.74 | 3.59 | 0.45 | 11 |
| es2 | 5.29 | 6.71 | 16.50 | 3.79 | 7 | es2 | 0.39 | 1.89 | 1.29 | 0.92 | 7 |
| es4 | 5.00 | 6.79 | 17.86 | 4.21 | 7 | es4 | 0.58 | 1.52 | 2.56 | 1.08 | 7 |
| es8 | 6.14 | 10.68 | 21.14 | 6.89 | 14 | es8 | 0.77 | 2.52 | 1.06 | 1.43 | 14 |
| mes | 6.30 | 10.25 | 22.25 | 6.33 | 10 | mes | 0.82 | 4.67 | 4.04 | 2.63 | 10 |

The described steps and materials in the foregoing examples are to be considered in all respects only as illustrative and not restrictive, and the scope of the invention is indicated by the appended claims rather than by the foregoing description. All changes which come within the meaning and range of equivalency of the claims are to be embraced within their scope.

### REFERENCES

Asker S E, L Jerling. 1992. Apomixis in plants. CRC Press, Boca Raton
Bala Ravi S. 1993. Apomixis in sorghum line R473—truth or myth? A critical analysis of published work. Cur Sci 64: 306-315
Bayer R J. 1989a. Patterns of isozyme variation in the Antennaria rosea (Asteraceae: Inuleae) polyploid agamic complex. Sys Bot 14: 389-397
Bayer R J. 1989b. A taxonomic revision of the Antennaria rosea (Asteraceae: Inuleae: Gnaphaliinae) polyploid complex. Brittonia 41: 53-60
Bayer R J. 1990. A phylogenetic reconstruction of Antennaria Gaertner (Asteraceae: Inuleae). Canad J Bot 68: 1389-1397
Bayer R J, B G Purdy, D G Lebedyk. 1991. Niche differentiation among eight sexual species of Antennaria Gaertner (Asteraceae: Inuleae) and A. rosea, their allopolyploid derivative. Evol Trends Plants 5: 109-123
Bayer R J, D E Soltis, P S Soltis. 1996. Phylogenetic inferences in Antennaria (Asteraceae: Gnaphalieae) based on sequences from the nuclear ribosomal DNA internal transcribed spacers (ITS). Amer J Bot 83: 516-527
Bayer R J, G L Stebbins. 1987. Chromosome numbers, patterns of distribution, and apomixis in Antennaria (Asteraceae: Inuleae). Sys Bot 12: 305-319
Bayer R J, T M Minish. 1993. Isozyme variation, ecology and phytogeography of Antennaria soliceps (Asteraceae: Inuleae), a geographically restricted alpine apomict from the Spring Mountains, Nevada. Madroño 40: 75-89
Carman J G. 1997. Asynchronous expression of duplicate genes in angiosperms may cause apomixis, bispory, tetraspory, and polyembryony. Biol J Linn Soc 61:51-94
Carman J G. 2000. The evolution of gametophytic apomixis. In: T Batygina ed: Embryology of Flowering Plants, Vol. 3: The Systems of Reproduction. Russian Academy of Sciences, St. Petersburg
Carman J G. 2001. The gene effect: genome collisions and apomixis. In: Y Savidan, J G Carman, T Dresselhaus eds: The Flowering of Apomixis: From Mechanisms to Genetic Engineering. Mexico, D. F.: CIMMYT, IRD, European Commission DG VI (FAIR)
Crane C F, J G Carman. 1987. Mechanisms of apomixis in Elymus rectisetus from Eastern Australia and New Zealand. Amer J Bot 74: 477-496
Esau K. 1977. Anatomy of Seed Plants. John Wiley & Sons, New York
Estrada-Luna A A, W Huanca-Mamani, G Acosta-Garcia, G Leon-Martinez, A Becerra-Flora, R1 Perez-Ruiz, J-PH Vielle-Calzada. 2002. Beyond promiscuity: from sexuality to apomixis in flowering plants. In Vitro Cell Devel Biol Plant 38:146-151
Grimanelli D, O Leblanc, E Perotti, U Grossniklaus. 2001. Developmental genetics of gametophytic apomixis. Trends Genet 17: 597-604
Grossniklaus U, GA Nogler, P J Van Dijk. 2001. How to avoid sex: the genetic control of gametophytic apomixis. Plant Cell 13: 1491-1498
Guillen-Portal F. R, D. D. Baltensperger, L. A. Nelson, G. Frickel, 2002, Assessment of hard red winter wheat F₂ and F₃ hybrids for the Nebraska Panhandle, Community Soil Science and Plant Analysis, Monticello, N.Y., Marcel Dekker Inc., 33 (5/6) p. 963-972
Gustafsson Å. 1946. Apomixis in higher plants. I. The mechanism of apomixis. Lunds Universitets Årsskrift 42: 1-67
Gustafsson Å. 1947a. Apomixis in higher plants. II. The causal aspect of apomixis. Lunds Universitets Årsskrift 43: 69-179
Gustafsson Å. 1947b. Apomixis in higher plants, III. Biotype and species formation. Lunds Universitets Årsskrift 43: 181-370
Hanna W W, K F Schertz, E C Bashaw. 1970. Apospory in Sorghum bicolor (L.) Moench. Science 170: 338-339
Juel O. 1900. Vergleichende untersuchungen über typische und parthenogenetische forpflanzung bei der gattung Antennaria. Kgl. Sven Vetenskapsakad, Ak. Handl. 33 No. 5: 1-59
Koltunow A M, U Grossniklaus. 2003. Apomixis: A developmental perspective. Annual Review of Plant Biology 54: 547-574
Leblanc O, M Dueñas, M Hernández, S Bello, V Garcia, J Berthaud, Y Savidan. 1995. Chromosome doubling in Tripsacum: the production of artificial, sexual tetraploid plants. Plant Breeding 114: 226-230
Nogler G A 1984. Gametophytic apomixis. In B M Johri ed, Embryology of Angiosperms, Berlin, Germany, Springer Verlag
Peel M D, J G Carman, O Leblanc. 1997a. Megasporocyte callose in apomictic buffelgrass, Kentucky bluegrass, Pennisetum squamulatum Fresen, Tripsacum L. and weeping lovegrass. Crop Sci 37: 724-732
Peel M D, J G Carman, Z W Liu, R R-C Wang. 1997b. Meiotic anomalies in hybrids between wheat and apomictic Elymus rectisetus (Nees in Lehm.) A. Löve & Connor. Crop Sci 37: 717-723
Poehlman J M. 1987. Breeding Field Crops. Van Nostrand Reinhold, New York
Ramsey J, D W Schemske. 1998. Pathways, mechanisms, and rates of polyploid formation in flowering plants. Ann Rev Ecol Syst 29: 467-501
Rana B S, C S Reddy, V J M Rao, N G P Rao. 1981. Apomixis in grain sorghums: analysis of seed set and effects of selection. Indian J Genet Plant Breed 41: 118-123
Richards A J. 2003. Apomixis in flowering plants: an overview. Philosophical Transactions of the Royal Society of London B 358: 1085-1093
Rieseberg L H, Widmer A, Arntz A M, Burke J M. 2003. The genetic architecture necessary for transgressive segregation is common in both natural and domesticated populations. Philosophical Transactions of the Royal Society of London B 358: 1141-1147.
Savidan Y. 2000. Apomixis: genetics and breeding. Plant Breed. Rev. 18: 13-86
Savidan Y H. 2001. Gametophytic apomixis: a successful mutation of the female gametogenesis. In S. S. Bhojwani, W Y Soh (ed), Current Trends in the Embryology of Angiosperms.
Schertz K F. 1992. Apomixis in sorghum. In: J H Elgin, J P Miksche eds, Proceedings of the Apomixis Workshop, Feb. 11-12, 1992, Atlanta, Ga., USDA-ARS, ARS-104
Sherwood R T. 2001. Genetic analysis of apomixis. In Y Savidan, J G Carman, T Dresselhaus (eds), The Flowering of Apomixis: From Mechanisms to Genetic Engineering, Mexico, D.F.: CIMMYT, IRD, European Commission DG VI (FAIR)
Spielman M, R Vinkenoog, R J Scott. 2003. Genetic mechanisms of apomixis, Phil Trans Royal Soc Lon B 358: 1095-1103
Tang C Y, K F Schertz, E C Bashaw. 1980. Apomixis in sorghum lines and their F₁ progenies. Bot Gaz 141: 294-299
Toenniessen G H. 2001. Feeding the world in the 21^{st} century: plant breeding, biotechnology, and the potential role of apomixis, In Y Savidan, J G Carman, T Dresselhaus (eds), The Flowering of Apomixis: From Mechanisms to Genetic Engineering, Mexico, D.F.: CIMMYT, IRD, European Commission DG VI (FAIR)
Weigel D, J. Glazebrook. 2002. Arabidopsis, A Laboratory Manual. Cold Spring Harbor Laboratory Press, Cold Spring Harbor, N.Y.
Yuan L P. 1993. Progress of two-line system in hybrid rice breeding, in: K Muralidhoran, E A Siddig (eds), New Frontiers in Rice Research, Hyderabad, India: Directorate of Rice Research, pg 86-93

What is claimed is:

1. A method of producing an apomictic progeny plant having an increased frequency of apomictic seed set comprising the steps of:
(a) obtaining a parent plant that expresses one or more elements of apomixis selected from the groups consisting of: unreduced embryo sac formation, parthenogenesis, adventitious embryony, pseudogamous endosperm formation, and autonomous endosperm formation and the parent plant is not genetically stable for the elements of apomixis, wherein the plant is a sorghum plant and the parent plant is obtained by:
identifying ecotypes or breeding lines from the breeding population that represent differences in germline development sequence (GDS) timing consisting of plants that have GDS stages that occur early relative to the maturity level of sporophytic ovule and ovary structures and plants that have GDS stages that occur late relative to the maturity level of sporophytic ovule and ovary structures; or plants that have GDS stages that occur early while others occur late relative to the maturity level of sporophytic ovule or ovary structure;
selecting from an ecotype or breeding line that represents extremes in GDS timing a first and second plant, wherein the mean onset time for embryo sac formation of the first plant occurs shortly after or before the mean onset time for megasporogenesis of the second plant relative to the maturity level of sporophytic ovule or ovary tissues;
hybridizing the first and second parent plants;
obtaining seed from the first or second plants;
sowing the seed obtained;
raising progeny plants there from; and
identifying plants that expresses elements of apomixis and are not genetically stable for the elements of apomixis to obtain the parent plant;
(b) self fertilizing the parent plant that expresses elements of apomixis, but is not genetically stable for the elements of apomixis;
(c) obtaining seed from the parent plants;
(d) sowing the seed obtained;
(e) raising progeny plants there from;
(f) screening the progeny plants for an increased frequency of apomictic seed set as compared to the parent plants; and
(g) isolating the progeny plant expressing the increased frequency of apomictic seed set of at least 5% greater than the parent plant.

2. The method of claim 1, wherein the frequency of apomictic seed set in the isolated progeny plant is at least 20% greater than the parent plants.

3. The method of claim 1, wherein the one or more elements of apomixis are unreduced embryo sac formation and parthenogenesis.

4. The method of claim 1, further comprising repeating steps (b) through (e) at least one time to obtain second generation or higher generation progeny having an increased frequency of apomictic seed set compared to the previous generation.

5. A method of producing a progeny plant that express a higher frequency of one or more elements of apomixis comprising the steps of:
(a) obtaining a parent plant that facultatively expresses elements of apomixis selected from the groups consisting of: unreduced embryo sac formation, parthenogenesis, adventitious embryony, pseudogamous endosperm formation, and autonomous endosperm formation, wherein the plant is a sorghum plant;
(b) self fertilizing the parent plant that facultatively expresses elements of apomixis;
(c) obtaining seed from the parent plants;
(d) sowing the seed obtained;
(e) raising progeny plants there from;
(f) screening the progeny plants for an increased frequency of expression of one or more elements of apomixis as compared to the parent plants; and
(g) isolating the progeny plants expressing increased frequency of one or more elements of apomixis of at least 5% greater than the parent plant.

6. The method of claim 5, wherein the one or more elements of apomixis are unreduced embryo sac formation and parthenogenesis.

7. The method of claim 5, further comprising repeating steps (b) through (e) at least one time to obtain second generation or higher generation progeny having an increased frequency of one or more elements of apomixis compared to the previous generation.

8. The method of claim 5, wherein the parent plant is obtained by:
identifying ecotypes or breeding lines from the breeding population that represent differences in germline development sequence (GDS) timing consisting of plants that have GDS stages that occur early relative to the maturity level of sporophytic ovule and ovary structures and plants that have GDS stages that occur late relative to the maturity level of sporophytic ovule and ovary structures; or plants that have GDS stages that occur early while others occur late relative to the maturity level of sporophytic ovule or ovary structure;
selecting from an ecotype or breeding line that represents extremes in GDS timing a first and second plant, wherein the mean onset time for embryo sac formation of the first plant occurs shortly after or before the mean onset time for megasporogenesis of the second plant relative to the maturity level of sporophytic ovule or ovary tissues;
hybridizing the first and second parent plants;
obtaining seed from the first or second plants;
sowing the seed obtained;
raising progeny plants there from; and
identifying plants that expresses elements of apomixis and are not genetically stable for the elements of apomixis to obtain the parent plant.

9. A method of producing an apomictic progeny plant having an increased frequency of apomictic seed set comprising the steps of:
(a) obtaining a parent plant that expresses one or more elements of apomixis and is not genetically stable for the elements of apomixis, wherein the plant is an Antennaria or Tripsacum plant and the one or more elements of apomixis is selected from the group consisting of: unreduced embryo sac formation, parthenogenesis, adventitious embryony, pseudogamous endosperm formation, and autonomous endosperm formation;
(b) self fertilizing the parent plant that expresses elements of apomixis, but is not genetically stable for the elements of apomixis;
(c) obtaining seed from the parent plants;
(d) sowing the seed obtained;
(e) raising progeny plants there from;
(f) screening the progeny plants for an increased frequency of apomictic seed set as compared to the parent plants; and
(g) isolating the progeny plant expressing the increased frequency of apomictic seed set, of at least 5% greater than the parent plant.

10. The method of claim 9, wherein the frequency of apomictic seed set in the isolated progeny plant is at least 20% greater than the parent plants.

11. The method of claim 10, wherein the one or more elements of apomixis are unreduced embryo sac formation and parthenogenesis.

12. The method of claim 9, further comprising repeating steps (b) through (e) at least one time to obtain second generation or higher generation progeny having an increased frequency of apomictic seed set compared to the previous generation.

13. The method of claim 9, wherein the parent plant is obtained by:
identifying ecotypes or breeding lines from the breeding population that represent differences in germline development sequence (GDS) timing consisting of plants that have GDS stages that occur early relative to the maturity level of sporophytic ovule and ovary structures and plants that have GDS stages that occur late relative to the maturity level of sporophytic ovule and ovary structures; or plants that have GDS stages that occur early while others occur late relative to the maturity level of sporophytic ovule or ovary structure;
selecting from an ecotype or breeding line that represents extremes in GDS timing a first and second plant, wherein the mean onset time for embryo sac formation of the first plant occurs shortly after or before the mean onset time for megasporogenesis of the second plant relative to the maturity level of sporophytic ovule or ovary tissues;
hybridizing the first and second parent plants;
obtaining seed from the first or second plants;
sowing the seed obtained;
raising progeny plants there from; and
identifying plants that expresses elements of apomixis and are not genetically stable for the elements of apomixis to obtain the parent plant.
